SEQUENCE LISTING (51 sequences)

SEQ ID NO: 1 - protein, 644 aa, Vaccinia virus
MEAVVNSDVFLTSNAGLKSSYTNQTLSLVDEDHIHTSDKSLSCSVCNSLSQIVDDDFISAGARNQRTKPKRAGNNQSQQPIKKDCMVSIDEVASTHDWSTRLRNDGNAIAKYLTTNKYDTSNFTIQDMLNIMNKLNIVRTNRNELFQLLTHVKSTLNNASVSVKCTHPLVLIHSRASPRIGDQLKELDKIYSPSNHHILLSTTRFQSMHFTDMSSSQDLSFIYRKPETNYYIHPILMALFGIKLPALENAYVHGDTYSLIQQLYEFRKVKSYNYMLLVNRLTEDNPIVITGVSDLISTEIQRANMHTMIRKAIMNIRMGIFYCNDDDAVDPHLMKIIHTGCSQVMTDEEQILASILSIVGFRPTLVSVARPINGISYDMKLQAAPYIVVNPMKMITTSDSPISINSKDIYSMAFDGNSGRVVFAPPNIGYGRCSGVTHIDPLGTNVMGSAVHSPVIVNGAMMFYVERRQNKNMFGGECYTGFRSLIDDTPIDVSPEIMLNGIMYRLKSAVCYKLGDQFFDCGSSDIFLKGHYTILFTENGPWMYDPLSVFNPGARNARLMRALKNQYKKLSMDSDDGFYEWLNGDGSVFAASKQQMLMNHVANFDDDLLTMEEAMSMISRHCCILIYAQDYDQYISARHITELF

SEQ ID NO: 2 - protein, 382 aa, Vaccinia virus
MDNLFTFLHEIEDRYARTIFNFHLISCDEIGDIYGLMKERISSEDMFDNIVYNKDIHHAIKKLVYCDIQLTKHIINQNTYPVFNDSSQVKCCHYFDINSDNSNISSRTVEIFEREKSSLVSYIKTTNKKRKVNYGEIKKTVHGGTNANYFSGKKSDEYLSTTVRSNINQPWIKTISKRMRVDIINHSIVTRGKSSILQTIEIIFTNRTCVKIFKDSTMHIILSKDKDEKGCIHMIDKLFYVYYNLFLLFEDIIQNEYFKEVANVVNHVLTATALDEKLFLIKKMAEHDVYGVSNFKIGMFNLTFIKSLDHTVFPSLLDEDSKIKFFKGKKLNIVALRSLEDCINYVTKSENMIEMMKERSTILNSIDIETESVDRLKELLLK

SEQ ID NO: 3 - protein, 1164 aa, Vaccinia virus
MKKNTDSEMDQRLGYKFLVPDPKAGVFYRPLHFQYVSYSNFILHRLHEILTVKRPLLSFKNNTERIMIEISNVKVTPPDYSPIIASIKGKSYDALATFTVNIFKEVMTKEGISITKISSYEGKDSHLIKIPLLIGYGNKNPLDTAKYLVPNVIGGVFINKQSVEKVGINLVEKITTWPKFRVVKPNSFTFSFSSVSPPNVLPTRYRHYKISLDISQLEALNISSTKTFITVNIVLLSQYLSRVSLEFIRRSLSYDMPPEVVYLVNAIIDSAKRITESITDFNIDTYINDLVEAEHIKQKSQLTINEFKYEMLHNFLPHMNYTPDQLKGFYMISLLRKFLYCIYHTSRYPDRDSMVCHRILTYGKYFETLAHDELENYIGNIRNDIMNNHKNRGTYAVNIHVLTTPGLNHAFSSLLSGKFKKSDGSYRTHPHYSWMQNISIPRSVGFYPDQVKISKMFSVRKYHPSQYLYFCSSDVPERGPQVGLVSQLSVLSSITNILTSEYLDLEKKICEYIRSYYKDDISYFETGFPITIENALVASLNPNMICDFVTDFRRRKRMGFFGNLEVGITLVRDHMNEIRINIGAGRLVRPFLVVDNGELMMDVCPELESRLDDMTFSDIQKEFPHVIEMVDIEQFTFSNVCESVQKFRMMSKDERKQYDLCDFPAEFRDGYVASSLVGINHNSGPRAILGCAQAKQAISCLSSDIRNKIDNGIHLMYPERPIVISKALETSKIAANCFGQHVTIALMSYKGINQEDGIIIKKQFIQRGGLDIVTAKKHQVEIPLENFNNKERDRSNAYSKLESNGLVRLNAFLESGDAMARNISSRTLEDDFARDNQISFDVSEKYTDMYKSRVERVQVELTDKVKVRVLTMKERRPILGDKFTTRTSQKGTVAYVADETELPYDENGITPDVIINSTSIFSRKTISMLIEVILTAAYSAKPYNNKGENRPVCFPSSNETSIDTYMQFAKQCYEHSNPKLSDEELSDKIFCEKILYDPETDKPYASKVFFGPIYYLRLRHLTQDKATVRCRGKKTKLIRQANEGRKRGGGIKFGEMERDCLIAHGAANTITEVLKDSEEDYQDVYVCENCGDIAAQIKGINTCLRCSKLNLSPLLTKIDTTHVSKVFLTQMNARGVKVKLDFERRPPSFYKPLDKVDLKPSFLV

SEQ ID NO: 4 - protein, 185 aa, Vaccinia virus
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 5 - protein, 204 aa, Vaccinia virus
MSRGALIVFEGLDKSGKTTQCMNIMESIPANTIKYLNFPQRSTVTGKMIDDYLTRKKTYNDHIVNLLFCANRWEFASFIQEQLEQGITLIVDRYAFSGVAYAAAKGASMTLSKSYESGLPKPDLVIFLESGSKEINRNVGEEIYEDVTFQQKVLQEYKKMIEEGDIHWQIISSEFEEDVKKELIKNIVIEAIHTVTGPVGQLWM

SEQ ID NO: 6 - protein, 552 aa, Vaccinia virus
MTSLREFRKLCCDIYHASGYKEKSKLIRDFITDRDDKYLIIKLLLPGLDDRIYNMNDKQIIKLYSIIFKQSQEDMLQDLGYGYIGDTIRTFFKENTEIRPRDKSILTLEDVDSFLTTLSSVTKESHQIKLLTDIASVCTCNDLKCVVMLIDKDLKIKAGPRYVLNAISPNAYDVFRKSNNLKEIIENSSKQNLDSISISVMTPINPMLAESCDSVNKAFKKFPSGMFAEVKYDGERVQVHKNNNEFAFFSRNMKPVLSHKVDYLKEYIPKAFKKATSIVLDSEIVLVDEHNVPLPFGSLGIHKKKEYKNSNMCLFVFDCLYFDGFDMTDIPLYERRSFLKDVMVEIPNRIVFSELTNISNESQLTDVLDDALTRKLEGLVLKDINGVYEPGKRRWLKIKRDYLNEGSMADSADLVVLGAYYGKGAKGGIMAVFLMGCYDDESGKWKTVTKCSGHDDNTLRVLQDQLTMIKINKDPKKIPEWLVVNKIYIPDFVVEDPKQSQIWEISGAEFTSSKSHTANGISIRFPRFTRIREDKTWKESTHLNDLVNLTKS

SEQ ID NO: 7 - protein, 151 aa, Vaccinia virus
MEREGVDYHYVNREAIWKGIAAGNFLEHTEFLGNIYGTSKTAVNTAAINNRICVMDLNIDGVRSFKNTYLMPYSVYIRPTSLKMVETKLRCRNTEANDEIHRRVILAKTDMDEANEAGLFDTIIIEDDVNLAYSKLIQILQDRIRMYFNTN

SEQ ID NO: 8 - protein, 353 aa, Vaccinia virus
MDIFKELIVKHPDENVLISPVSILSTLSILNHGAAGSTAEQLSKYIENMNENTPDDNNDMDVDIPYCATLATANKIYGSDSIEFHASFLQKIKDDFQTVNFNNANQTKELINEWVKTMTNGKINSLLTSPLSINTRMTVVSAVHFKAMWKYPFSKHLTYTDKFYISKNIVTSVDMMVGTENNLQYVHINELFGGFSIIDIPYEGNSSMVIILPDDIEGIYNIEKNITDEKFKKWCGMLSTKSIDLYMPKFKVEMTEPYNLVPILENLGLTNIFGYYADFSKMCNETITVEKFLHTTFIDVNEEYTEASAVTGVFTINFSMVYRTKVYINHPFMYMIKDTTGRILFIGKYCYPQ

SEQ ID NO: 9 - protein, 844 aa, Vaccinia virus
MDANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNEKVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTKNFFELNRGAIKCEGLDVEDLLSYYVVYVFSKR

SEQ ID NO: 10 - protein, 785 aa, Vaccinia virus
MDAAIRGNDVIFVLKTIGVPSACRQNEDPRFVEAFKCDELKRYIDNNPECTLFESLRDEEAYSIVRIFMDVDLDACLDEIDYLTAIQDFIIEVSNCVARFAFTECGAIHENVIKSMRSNFSLTKSTNRDKTSFHIIFLDTYTTMDTLIAMKRTLLELSRSSENPLTRSIDTAVYRRKTTLRVVGTRKNPNCDTIHVMQPPHDNIEDYLFTYVDMNNNSYYFSLQRRLEDLVPDKLWEPGFISFEDAIKRVSKIFINSIINFNDLDENNFTTVPLVIDYVTPCALCKKRSHKHPHQLSLENGAIRIYKTGNPHSCKVKIVPLDGNKLFNIAQRILDTNSVLLTERGDYIVWINNSWKFNSEEPLITKLILSIRHQLPKEYSSELLCPRKRKTVEANIRDMLVDSVETDTYPDKLPFKNGVLDLVDGMFYSGDDAKKYTCTVSTGFKFDDTKFVEDSPEMEELMNIINDIQPLTDENKKNRELYEKTLSSCLCGATKGCLTFFFGETATGKSTTKRLLKSAIGDLFVETGQTILTDVLDKGPNPFIANMHLKRSVFCSELPDFACSGSKKIRSDNIKKLTEPCVIGRPCFSNKINNRNHATIIIDTNYKPVFDRIDNALMRRIAVVRFRTHFSQPSGREAAENNDAYDKVKLLDEGLDGKIQNNRYRFAFLYLLVKWYKKYHVPIMKLYPTPEEIPDFAFYLKIGTLLVSSSVKHIPLMTDLSKKGYILYDNVVTLPLTTFQQKISKYFNSRLFGHDIESFINRHKKFANVSDEYLQYIFIEDISSP

SEQ ID NO: 11 - protein, 190 aa, Vaccinia virus
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDANPVTIINEYCQITKRDWSFRIESVGPSNSPTFYACVDIDGRVFDKADGKSKRDAKNNAAKLAVDKLLGYVIIRF

SEQ ID NO: 12 - protein, 49 aa, Vaccinia virus
MVIGLVIFVSVAAAIVGVLSNVLDMFMYVEENNEEDARIKEEQELLLLY

SEQ ID NO: 13 - protein, 635 aa, Vaccinia virus
MLNRVQILMKTANNYETIEILRNYLRLYIILARNEEGHGILIYDDNIDSIMSMMNITRLEVIGLTTHCTKLRSSPPIPMSRLFMDEIDHESYYSPKTSDYPLIDIIRKRSHEQGDIALALEQYGIENTDSISEINEWLSSKGLACYRFVKFNDYRKQMYRKFSRCTIVDSMIIGHIGHHYIWIKNLETYTRPEIDVLPFDIKYISRDELWVRISSSLDQTHIKTIAVSVYGAITDNGPIPYMISTYPGNTFVNFNSVKNLILNFLDWIKDIMTSTRTIILVGYMSNLFDIPLLTVYWPNNCGWKIYNNTLISSDGARVIWMDAYKFSCGLSLQDYCYHWGSKPESRPFDLIKKSDAKRNSKSLVKESMASLKSLYEAFETQSGALEVLMSPCRMFSFSRIEDMFLTSVINRVSENTGMGMYYPTNDIPSLFIESSICLDYIIVNNQESNKYRIKSVLDIISSKQYPAGRPNYVKNGTKGKLYIALCKVTVPTNDHIPVVYHDDDNTTTFITVLTSVDIETAIRAGYSIVELGALQWDNNIPELKNGLLDSIKMIYDLNAVTTNNLLEQLIENINFNNSSIISLFYTFAISYCRAFIYSIMETIDPVYISQFSYKELYVSSSYKDINESMSQMVKL

SEQ ID NO: 14 - protein, 269 aa, Vaccinia virus
MSKVIKKRVETSPRPTASSDSLQTCAGVIEYAKSISKSNAKCIEYVTLNASQYANCSSISIKLTDSLSSQMTSTFIMLEGETKLYKNKSKQDRSDGYFLKIKVTAASPMLYQLLEAVYGNIKHKERIPNSLHSLSVETITEKTFKDESIFINKLNGAMVEYVSAGESSILRSIEGELESLSKRERQLAKAIITPIVFYRSGTETKITFALKKLIIDREVVANVIGLSGDSERVSMTENVEEDLARNLGLVDIDDEYDEDSDKEKPIFNV

SEQ ID NO: 15 - protein, 126 aa, Vaccinia virus
MRILFLIAFMYGCVHPYVNADEIKCPNLNIVTSSGEFRCTGCVKFMPNFSYMYWLAKDMRSDEDAKFIEHLGEGIKEDETVSTIDGRIVTLQKVLHVTDTNKFDNYRFTCVLTTIDGVSKKNIWLK

SEQ ID NO: 16 - protein, 67 aa, Vaccinia virus
RSDEDTKFIEHLGDGIKEDETVRTTDSGITTLRKVLHVTDTNKFAHYRFTCVLTTIDGVSKKNIWLK

SEQ ID NO: 17 - protein, 250 aa, Vaccinia virus
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMVIATTDMQN

SEQ ID NO: 18 - protein, 220 aa, Vaccinia virus
MVYKLVLLFCIASLGYSVEYKNTICPPRQDYRYWYFAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYGMRGSYGVTFIDELNQCLLDIKELSYDICYRE

SEQ ID NO: 19 - DNA, 14 bp, Artificial
  synthetic dsDNA Adaptor
gagggtccgacagc

SEQ ID NO: 20 - DNA, 24 bp, Artificial
  synthetic primer
ggcgctagcatggccgtcatggcg

SEQ ID NO: 21 - DNA, 34 bp, Artificial
  synthetic primer
ggcctcgagtcacactttacaagctgtgagagac

SEQ ID NO: 22 - protein, 9 aa, Vaccinia virus
DEVASTHDW

SEQ ID NO: 23 - protein, 9 aa, Vaccinia virus
YEFRKVKSY

SEQ ID NO: 24 - protein, 9 aa, Vaccinia virus
HDVYGVSNF

SEQ ID NO: 25 - protein, 9 aa, Vaccinia virus
ITDFNIDTY

SEQ ID NO: 26 - protein, 9 aa, Vaccinia virus
TYNDHIVNL

SEQ ID NO: 27 - protein, 9 aa, Vaccinia virus
WLKIKRDYL

SEQ ID NO: 28 - protein, 9 aa, Vaccinia virus
VYINHPFMY

SEQ ID NO: 29 - protein, 9 aa, Vaccinia virus
EERHIFLDY

SEQ ID NO: 30 - protein, 9 aa, Vaccinia virus
LENGAIRIY

SEQ ID NO: 31 - protein, 9 aa, Vaccinia virus
EEIPDFAFY

SEQ ID NO: 32 - protein, 9 aa, Vaccinia virus
VWINNSWKF

SEQ ID NO: 33 - protein, 9 aa, Vaccinia virus
DDVSREKSM

SEQ ID NO: 34 - protein, 9 aa, Vaccinia virus
EEQELLLLY

SEQ ID NO: 35 - protein, 9 aa, Vaccinia virus
IEGELESLS

SEQ ID NO: 36 - protein, 9 aa, Vaccinia virus
DEIKCPNLN

SEQ ID NO: 37 - protein, 9 aa, Vaccinia virus
AELTIGVNY

SEQ ID NO: 38 - protein, 9 aa, Molluscum contagiosum virus
DEVASTQDW

SEQ ID NO: 39 - protein, 9 aa, Molluscum contagiosum virus
YELKKVRPD

SEQ ID NO: 40 - protein, 10 aa, Molluscum contagiosum virus
AHMYYGVHNF

SEQ ID NO: 41 - protein, 9 aa, Molluscum contagiosum virus
EDDFDVAEY

SEQ ID NO: 42 - protein, 9 aa, Molluscum contagiosum virus
EEQYVFLDF

SEQ ID NO: 43 - protein, 9 aa, Molluscum contagiosum virus
LGNGALRIF

SEQ ID NO: 44 - protein, 9 aa, Molluscum contagiosum virus
DLIPDFCFQ

SEQ ID NO: 45 - protein, 9 aa, Molluscum contagiosum virus
VWLRNCWRF

SEQ ID NO: 46 - protein, 9 aa, Molluscum contagiosum virus
MLRELETLA

SEQ ID NO: 47 - protein, 9 aa, Variola virus
VETKCPNLD

SEQ ID NO: 48 - protein, 9 aa, Monkeypox virus
VETKCPNLA

SEQ ID NO: 49 - protein, 11 aa, Vaccinia virus
KIQNVIIDECY

SEQ ID NO: 50 - protein, 20 aa, Vaccinia virus
NPITKTTSDYQDSDVSQEVR

SEQ ID NO: 51 - protein, 14 aa, Vaccinia virus
TKTTSDYQDSDVSQ

(19) United States
(12) Patent Application Publication
(10) Pub. No.: US 2009/0269365 A1
(43) Pub. Date: Oct. 29, 2009

(54) IMMUNOGENIC VACCINIA PEPTIDES AND METHODS OF USING SAME

(75) Inventors: David M. Koelle, Seattle, WA (US); Lichen Jing, Seattle, WA (US)

Correspondence Address: KAREN S. CANADY;CANADY & LORTZ LLP, 4201 Wilshire BLV Suite 622, LOS ANGELES, CA, 90010, US

(73) Assignee: UNIVERSITY OF WASHINGTON, Seattle, WA (US)

(21) Appl. No.: 11/911,706

(22) PCT Filed: Apr. 20, 2006
(86) PCT No.: PCT/US2006/015307

§ 371 Date: Oct. 16, 2007

Related U.S. Application Data
(60) Provisional application No. 60/673,266, filed on Apr. 20, 2005.
(60) Provisional application No. 60/714,458, filed on Sep. 6, 2005.

(51) Int. Cl. A61K 39/12; A61K 38/18; C07H 21/04; C12N 15/74; C12N 5/02; A61K 31/7088; C12N 7/00; A61K 45/00; A61P 31/00

(52) U.S. Cl. 424/186.1; 514/12; 536/23.72; 435/320.1; 435/325; 514/44 R; 435/235.1; 424/93.2

(57) ABSTRACT

The invention provides specific proteins encoded by the vaccinia genome that elicit an immune memory response and can be used for vaccines directed against variola (smallpox), monkeypox and other poxviruses. The invention provides antigens, polypeptides comprising antigens, polynucleotides encoding the polypeptides, vectors, and recombinant viruses containing the polynucleotides, antigen-presenting cells (APCs) presenting the polypeptides, immune cells directed against the epitopes, and pharmaceutical compositions. The invention additionally provides methods, including methods for preventing and treating infection, for killing infected cells, for inhibiting viral replication, for enhancing secretion of antiviral and/or immunomodulatory lymphokines, and for enhancing production of disease-specific antibody.

### CROSS-REFERENCE TO RELATED APPLICATIONS

This application claims benefit of U.S. provisional patent application Nos. 60/673,266, filed Apr. 20, 2005, and 60/714,458, filed Sep. 6, 2005, the entire contents of each of which are incorporated herein by reference.

### STATEMENT REGARDING FEDERALLY SPONSORED RESEARCH

This invention was made with government support under grant number R21 AI061636 awarded by the National Institutes of Health. The government may have certain rights in this invention.

### TECHNICAL FIELD OF THE INVENTION

The invention relates to molecules, compositions and methods that can be used for the treatment and prevention of viral infection and other diseases. More particularly, the invention identifies epitopes of vaccinia proteins that can be used for methods, molecules and compositions having the antigenic specificity of vaccinia-specific T cells, and in particular, of, CD4+ and CD8+ T cells. In addition, the invention relates to a method for testing and identifying further epitopes useful in the development of diagnostic and therapeutic agents for detecting, preventing and treating viral infection and other diseases.

### BACKGROUND OF THE INVENTION

Vaccinia are a set of closely related orthopox viruses. Variola and monkeypox are also orthopox viruses. Variola causes the deadly disease smallpox. There is increased concern about smallpox as a bioterrorism agent. Monkeypox causes disease in primates and other animals and occasionally causes disease in humans. Purposeful inoculation with live vaccinia leads to mild, transitory infection. The immune memory provoked by vaccinia infection then either prevents smallpox infection from occurring, or renders smallpox infection harmless. Infection with the strain of vaccinia used in the United States, Dryvax™ marketed by Wyeth, as well as other strains used in other parts of the world, has toxic side effects in some persons, creating a need for safer alternative vaccines that can also provoke an immunologic memory to prevent or ameliorate smallpox infection.

Vaccinia is a relatively avirulent orthopoxirus that stimulates cross-protective immunity against variola. Very little is known about the specific CD4 and CD8 T cell response induced by vaccinia. There remains a need for detailed information about the poxvirus antigens and epitopes recognized by CD4 and CD8 T-cells, to understand how vaccinia works, and to develop new candidate vaccines for the prevention of variola. More specifically, there remains a need to identify epitopes capable of eliciting an effective immune response to variola infection. Such information can lead to the identification of more effective immunogenic antigens and/or safer vaccines useful for the prevention and treatment of smallpox and other orthopox virus infections.

### SUMMARY OF THE INVENTION

The invention provides specific proteins encoded by the vaccinia genome that elicit an immune memory response. The invention provides antigens, polypeptides comprising antigens, polynucleotides encoding the polypeptides, vectors, and recombinant viruses containing the polynucleotides, antigen-presenting cells (APCs) presenting the polypeptides, immune cells directed against the epitopes, and pharmaceutical compositions. The pharmaceutical compositions can be used both prophylactically and therapeutically. The invention additionally provides methods, including methods for preventing and treating infection, for killing infected cells, for inhibiting viral replication, for enhancing secretion of antiviral and/or immunomodulatory lymphokines, and for enhancing production of disease-specific antibody. The method comprises administering to a subject an effective amount of a polypeptide, polynucleotide, recombinant virus, APC, immune cell or composition of the invention. The methods for killing infected cells and for inhibiting viral replication comprise contacting an infected cell with an immune cell of the invention. The immune cell of the invention is one that has been stimulated by an antigen of the invention or by an APC that presents an antigen of the invention. A method for producing such immune cells is also provided by the invention. The method comprises contacting an immune cell with an APC, preferably a dendritic cell, that has been modified to present an antigen of the invention. In a preferred embodiment, the immune cell is a T cell such as a CD4+ or CD8+ T cell.

The diseases to be prevented or treated using compositions and methods of the invention include diseases associated with orthopox virus infection. Examples of orthopox viruses include cowpox, camelpox,, monkeypox, variola (smallpox), and ectromelia (mice). Variola and monkeypox are the pathogens of particular concern for humans. Examples of vaccinia antigens that have been identified by the method of the invention include A3L, A23R, A24R, A33R, A48R, A50R, A57R, C12L, D1R, D5R, E3L, F3, F12L, I3L, IL18bp, IL-18bp-like protein, L1R, or M2L. In addition, immunologically active fragments within these vaccinia proteins have been identified and are listed in the appendix. The epitopes described herein can be used in the preparation of subunit vaccines for prevention of smallpox and monkeypox and other orthopox-associated diseases. In addition, the epitopes of the invention provide reagents for immunogenicity testing of candidate smallpox (and other orthopox) vaccines.

The invention further provides a method of testing reagents for immunogenicity for vaccinia-based vaccines for other indications such as HIV, malaria, and cancer. Those skilled in the art are familiar with methods for introducing foreign genes (microbial, cancer-related) into vaccinia by genetic engineering and then injecting these into patients to stimulate an immune response against the foreign gene. One can modify the vaccinia vector backbones, for example, by inserting pro-immunogenicity genes like cytokines or adhesion molecules into vaccinia (in addition to the disease-associated gene, such as an HIV or cancer gene). To compare various vector backbones, diagnostic tests of immune responses against these vaccinia CD8 epitopes can be used.

The invention additionally provides pharmaceutical compositions comprising the vaccinia antigens and epitopes identified herein. Also provided is an isolated polynucleotide that encodes a polypeptide of the invention, and a composition comprising the polynucleotide. The invention additionally provides a recombinant virus genetically modified to express a polynucleotide of the invention, and a composition comprising the recombinant virus. In one embodiment, the recombinant virus is an adenovirus or alphavirus. A composition of the invention can be a pharmaceutical composition. The composition can optionally comprise a pharmaceutically acceptable carrier and/or an adjuvant.

### BRIEF DESCRIPTION OF THE DRAWINGS

FIG. 1A & 1B: Detection of vaccinia-specific CD8 lymphocytes in PBMC. 1A, Intracellular cytokine cytometry. PBMC from before or 4 wk after primary intradermal Dryvax were stimulated with live vaccinia for 6 h. The proportion of CD8+ lymphocytes staining positive for IFN-γ is indicated. Staining with an isotype control is also shown. 1B, Vaccinia-specific CD8 CTL activity is present in human PBMC after intradermal vaccination with Dryvax. After one cycle of restimulation in vitro, CD8+ cells were purified for ⁵¹Cr CTL assays at an E:T ratio of 20. Allogeneic target cells were HLA class I-mismatched.

FIG. 2: Clones with cytotoxic activity toward autologous vaccinia-infected LCL but not mock-infected LCL are readily derived from CD8 cells purified from PBMC stimulated with live vaccinia. Subject numbers, weeks after vaccination, and the number of clones screened are indicated. Data are percent-specific release from ⁵¹Cr CTL assays of candidate clones. Subject 2 is a primary vaccinee and the other subjects are revaccinees. Clones in the upper left quadrants with >20% killing of infected targets and <10% killing of uninfected targets were considered positive.

FIG. 3A & 3B: Representative example of cytotoxicity and transfection/infection tests to establish HLA restriction. 3A, ⁵¹Cr CTL assays for clone 2.59 from a primary vaccinee vs. autologous, fully mismatched, or partially HLA class I-matched (matching alleles indicated) LCL targets with or without vaccinia infection. 3B, IFN-γ release by clone 2.59 after coincubation with Cos-7 cells transfected with HLA B*4403 cDNA, infection with vaccinia, or both. Controls at right are coincubation with autologous LCL. Data are means of triplicate assays.

FIG. 4: Analysis of vaccinia genomic library plasmids that stimulated IFN-γ release by CD8 CTL clone 2.59. Top line, Genomic structure of vaccinia Copenhagen with common and systematic gene nomenclatures. P, Selected sequences with vaccinia early promoter features. ATG, Methionine codons M1 and M25 within F3. SOR, Shortest overlapping region of positive library plasmids. The sequence of ORF F3 25-49 is shown at the bottom, with the B*4403-restricted epitope underlined.

FIG. 5: Vaccinia-specific CD8 clones recognize synthetic peptides at low concentrations. Legend indicates the clone, HLA restriction, ORF, and amino acid residues in nonamer peptides. Autologous LCL were peptide-loaded, washed, and used in standard ⁵¹Cr CTL assays.

FIG. 6A & 6B: Recognition of vaccinia protein fragments by bulk vaccinia-specific CTL from subject 2 (6A and 6B left) and subject 5 (6B right, A*0101). Cos-7 were transfected with the indicated plasmids as fusions with eGFP-C1, with or without the indicated HLA cDNAs. IFN-γ release is indicated by the mean and SD of duplicate OD450 readings. Controls are Cos-7 untransfected or transfected with HLA cDNA only.

FIG. 7A & 7B: Recognition of vaccinia peptides by bulk vaccinia-specific T cells from the indicated subjects. Cells were stimulated for 15 h with 1 μM peptide or DMSO as per Materials and Methods, permeabilized and stained with anti-IFN-y-PE or isotype. 7A left, CD8+ cells purified from bulk CTL were tested with DMSO or representative positive and negative peptides selected from the sequence genomic library fragments that were active with the indicated HLA cDNAs. Data are the proportion of cells (R2 plus R3 gate) with high isotype control or IFN-γ signal (R3). 7B, Bulk CTL stimulated with DMSO, previously reported A*0201 epitopes or the A50R 395-403 peptide, and stained as above, or stained with anti-CD8+ and specific B*0801/A50R 395-403 tetramer (WLK) or a control tetramer (RPR). The percentage of CD8+ cells in the right upper quadrant is shown.

FIG. 8: Recognition of vaccinia peptides by bulk vaccinia-specific CD8 CTL using IFN-γ release as the readout. Autologous LCL were pulsed with the indicated representative peptides and concentrations, washed, and coincubated with bulk CTL. Supernatants were assayed for IFN-γ release. Values are means of duplicates.

### DETAILED DESCRIPTION OF THE INVENTION

### Definitions

All scientific and technical terms used in this application have meanings commonly used in the art unless otherwise specified. As used in this application, the following words or phrases have the meanings specified.

As used herein, “polypeptide” includes proteins, fragments of proteins, and peptides, whether isolated from natural sources, produced by recombinant techniques or chemically synthesized. Polypeptides of the invention typically comprise at least about 6 amino acids, and can be at least about 15 amino acids. Typically, optimal immunological potency is obtained with lengths of 8-10 amino acids. Those skilled in the art also recognize that additional adjacent sequence from the original (native) protein can be included, and is often desired, in an immunologically effective polypeptide suitable for use as a vaccine. This adjacent sequence can be from 1, 2, 3, 4, 5, 6, 7, 8, 9 or 10 amino acids in length to as much as 15, 20, 25, 30, 35, 40, 45, 50, 75 or 100 amino acids in length or more.

As used herein, particularly in the context of polypeptides of the invention, “consisting essentially of” means the polypeptide consists of the recited amino acid sequence and, optionally, adjacent amino acid sequence. The adjacent sequence typically consists of additional, adjacent amino acid sequence found in the full length antigen, but variations from the native antigen can be tolerated in this adjacent sequence while still providing an immunologically active polypeptide.

As used herein, “epitope” refers to a molecular region of an antigen capable of eliciting an immune response and of being specifically recognized by the specific immune T-cell produced by such a response. Another term for “epitope” is “determinant” or “antigenic determinant”. Those skilled in the art often use the terms epitope and antigen interchangeably in the context of referring to the determinant against which an immune response is directed.

As used herein, “vaccinia” includes any strain of vaccinia, unless otherwise indicated. References to amino acids of vaccinia proteins or polypeptides are based on the genomic sequence information regarding vaccinia Copenhagen as described in Goebel, S. J., et al., The complete DNA sequence of vaccinia virus, Virology 179 (1), 247-266 (1990) and having GenBank Accession No. NC_{—}001559, unless otherwise indicated. For the antigen VACWR013, also known as IL-18bp, the Copenhagen strain lacks a corresponding ORF. The genomic sequence for vaccinia strain WR (VACWR) is described in GenBank Accession No. AY243312.

As used herein, “substitutional variant” refers to a molecule having one or more amino acid substitutions or deletions in the indicated amino acid sequence, yet retaining the ability to be “immunologically active”, or specifically recognized by an immune cell. The amino acid sequence of a substitutional variant is preferably at least 80% identical to the native amino acid sequence, or more preferably, at least 90% identical to the native amino acid sequence. Typically, the substitution is a conservative substitution.

One method for determining whether a molecule is “immunologically active”, “immunologically effective”, or can be specifically recognized by an immune cell, is the cytotoxicity assay described in D. M. Koelle et al., 1997, Human Immunol. 53:195-205. Other methods for determining whether a molecule can be specifically recognized by an immune cell are described in the examples provided hereinbelow, including the ability to stimulate secretion of interferon-gamma or the ability to lyse cells presenting the molecule. An immune cell will specifically recognize a molecule when, for example, stimulation with the molecule results in secretion of greater interferon-gamma than stimulation with control molecules. For example, the molecule may stimulate greater than 5 pg/ml, or preferably greater than 10 pg/ml, interferon-gamma secretion, whereas a control molecule will stimulate less than 5 pg/ml interferon-gamma.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “adjuvant” includes those adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.; Merck Adjuvant 65 (Merck and Company, Inc., Rahway, N.J.); AS-2 (Smith-Kline Beecham); QS-21 (Aquilla); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

As used herein, to “prevent” or “protect against” a condition or disease means to hinder, reduce or delay the onset or progression of the condition or disease.

### Overview

Vaccinia infection provokes strong cytotoxic T-lymphocyte (CTL) responses. In mice, these CTL are mostly CD8+ cells. The response is large: 22-25% of CD8+ splenocytes are vacciria-reactive at 7 days, declining to 4-5% at 1-3 months. In humans, the magnitude of the primary CD8 response has been measured at ˜1%. Cytotoxicity, interferon-γ (IFN-γ), and tumor necrosis factor-α (TNF-α) responses are readily detectable. The human CD8 CTL data described in the Examples below are also consistent with brisk primary induction of virus-specific CD8 cells. The vaccinia-specific CD8+ CTL clones described herein make large amounts of IFN-γ in response to vaccinia.

Vaccinia has a ˜200 kB genome. The complete genome sequence of Vaccinia virus, Copenhagen strain, has been deposited with Genbank, Accession No. NC_{—}001559 and has a total of 191737 bp in this sequence. The sequences of other strains and other orthopox viruses can be found via the website maintained by poxvitus.org. Throughout this document, references to amino acids of vaccinia proteins or polypeptides are based on the genomic sequence information regarding vaccinia Copenhagen as described in Genbank Accession No. NC_{—}001559 and published in Goebel, S. J., et al., The complete DNA sequence of vaccinia virus, Virology 179 (1), 247-266 (1990).

### Vaccinia Polypeptides

In one embodiment, the invention provides an isolated vaccinia polypeptide. The polypeptide comprises a A3L, A23R, A24R, A33R, A48R, A50R, A57R, C12L, D1R, D5R, E3L, F3, F12L, I3L, IL-18bp, IL-18bp-like protein, L1R, or M2L protein or a fragment thereof. In some representative embodiments, the fragment comprises amino acids:
42-118, 90-98, 213-304, 273-304, 264-272, 392-474, 393-474, 487-567 of A3L;
259-376, 287-295 of A23R;
108-338, 267-339, 246-480, 246-339, 278-286, 747-897 of A24R;
160-173, 157-176, 58-185 of A33R;
58-66, 55-119, 55-120, 1-132, 1-133, 53-134, 54-136 of A48R;
395-403, 359-439 of A50R;
1-62 of A57R;
301-353, 326-334, 320-353 of C12L;
126-134, 47-158 of D1R;
208-397, 214-397, 349-357, 290-391, 298-306, 606-760, 618-760, 691-699 of D5R;
41-123, 55-123, 86-94 of E3L;
41-49, 1-49, 25-49, 26-49 of F3;
147-280, 392-386, 392-486 of F12L;
53-206, 109-197, 118-257, 118-197, 116-192, 173-181 of I3L;
1-41, 1-51, 21-29, 59-126 of IL-18bp;
59-126 of IL-18bp-like protein;
1-185, 127-137 of L1R;
24-172, or 38-46 of M2L.
A list of fragments containing antigenic regions can be found in Example 3 below.

A fragment of the invention consists of less than the complete amino acid sequence of the corresponding protein, but includes the recited epitope or antigenic region. As is understood in the art and confirmed by assays conducted using fragments of widely varying lengths, additional sequence beyond the recited epitope can be included without hindering the immunological response. A fragment of the invention can be as few as 8 amino acids in length, or can encompass 10%, 20%, 30%, 40%, 50%, 60%, 70%, 80%, 90% or 95% of the full length of the protein.

The optimal length for the polypeptide of the invention will vary with the context and objective of the particular use, as is understood by those in the art. In some vaccine contexts, a full-length protein or large portion of the protein (e.g., up to 100 amino acids, 150 amino acids, 200 amino acids, 250 amino acids or more) provides optimal immunological stimulation, while in others, a short polypeptide (e.g., less than 50 amino acids, 40 amino acids, 30 amino acids, 20 amino acids, 15 amino acids or fewer) comprising the minimal epitope and/or a small region of adjacent sequence facilitates delivery and/or eases formation of a fusion protein or other means of combining the polypeptide with another molecule or adjuvant.

A polypeptide for use in a composition of the invention comprises a vaccinia polypeptide that contains an epitope or minimal stretch of amino acids sufficient to elicit an immune response. These polypeptides typically consist of such an epitope and, optionally, adjacent sequence. Those skilled in the art are aware that the vaccinia epitope can still be immunologically effective with a small portion of adjacent vaccinia or other amino acid sequence present. Accordingly, a typical polypeptide of the invention will consist essentially of the recited vaccinia epitope and have a total length of up to 15, 20, 25 or 30 amino acids.

A3L (VACVgp154)

(SEQ ID NO: 1)

MEAVVNSDVFLTSNAGLKSSYTNQTLSLVDEDHIHTSDKSLSCSVCNSLS
QIVDDDFISAGARNQRTKPKRAGNNQSQQPIKKDCMVSIDEVASTHDWST
RLRNDGNAIAKYLTTNKYDTSNFTIQDMLNIMNKLNIVRTNRNELFQLLT
HVKSTLNNASVSVKCTHPLVLIHSRASPRIGDQLKELDKIYSPSNHHILL
STTRFQSMHFTDMSSSQDLSFIYRKPETNYYIHPILMALFGIKLPALENA
YVHGDTYSLIQQLYEFRKVKSYNYMLLVNRLTEDNPIVITGVSDLISTEI
QRANMHTMIRKAIMNIRMGIFYCNDDDAVDPHLMKIIHTGCSQVMTDEEQ
ILASILSIVGFRPTLVSVARPINGISYDMKLQAAPYIVVNPMKMITTSDS
PISINSKDIYSMAFDGNSGRVVFAPPNIGYGRCSGVTHIDPLGTNVMGSA
VHSPVIVNGAMMFYVERRQNKNMFGGECYTGFRSLIDDTPIDVSPEIMLN
GIMYRLKSAVCYKLGDQFFDCGSSDIFLKGHYTILETENGPWMYDPLSVF
NPGARNARLMRALKNQYKKLSMDSDDGFYEWLNGDGSYFAASKQQMLMNH
VANFDDDLLTMEEAMSMISRHCCI LIYAQDYDQYISARHITELF
A23R (VACVgp183)

(SEQ ID NO: 2)

MDNLFTFLHEIEDRYARTIFNFHLISCDEIGDIYGLMKERISSEDMFDNI
VYNKDIHHAIKKLVYCDIQLTKHIINQNTYPVFNDSSQVKCCHYFDINSD
NSNISSRTVEIFEREKSSLVSYIKTTNKKRKVNYGEIKKTVHGGTNANYF
SGKKSDEYLSITVRSNINQPWIKTISKRMRVDIINHSIVTRGKSSILQTI
EIIFTNRTCVKIFKDSTMHIILSKDKDEKGCIHMIDKLFYVYYNLFLLFE
DIIQNEYFKEVANVVNHVLTATALDEKLFLIKKMAEHDVYGVSNFKIGMF
NLTFIKSLDHTVFPSLLDEDSKIKFFKGKKLNIVALRSLEDCINYVTKSE
NMIEMMKERSTILNSIDIETESVDRLKELLLK
A24R (VACVgp184)

(SEQ ID NO: 3)

MKKNTDSEMDQRLGYKFLVPDPKAGVFYRPLHFQYVSYSNFILHRLHEIL
TVKRPLLSFKNNTERIMIEISNVKVTPPDYSPIIASIKGKSYDALATFTV
NIFKEVMTKEGISITKISSYEGKDSHLIKIPLLIGYGNKNPLDTAKYLVP
NVIGGVFINKQSVEKVGINLVEKITTWPKFRVVKPNSFTFSFSSVSPPNV
LPTRYRHYKISLDISQLEALNISSTKTFITVNIVLLSQYLSRVSLEFIRR
SLSYDMPPEVVYLVNAIIDSAKRITESITDFNIDTYINDLVEAEHIKQKS
QLTINEFKYEMLHNFLPHMNYTPDQLKGFYMISLLRKFLYCIYHTSRYPD
RDSMVCHRILTYGKYFETLAHDELENYIGNIRNDIMNNHKNRGTYAVNIH
YLTTPGLNHAFSSLLSGKFKKSDGSYRTHPHYSWMQNISIPRSVGFYPDQ
VKISKMFSVRKYHPSQYLYFCSSDVPERGPQVGLVSQLSVLSSITNILTS
EYLDLEKKICEYIRSYYKDDISYFETGFPITIENALVASLNPNMICDFVT
DFRRRKRMGFFGNLEVGITLVRDHMNEIRINIGAGRLVRPFLVVDNGELM
NDVCPELESRLDDMTFSDIQKEFPHVIEMYDIEQFTFSNVCESVQKFRMM
SKDERKQYDLCDFPAEFRDGYVASSLVGINHNSGPRAILGCAQAKQAISC
LSSDIRNKIDNGIHLMYPERPIVISKALETSKIAANCFGQHVTIALMSYK
GINQEDGIIIKKQFIQRGGLDIVTAKKHQVEIPLENFNNKERDRSNAYSK
LESNGLVRLNAFLESGDAMARNISSRTLEDDFARDNQISFDVSEKYTDMY
KSRVERVQVELTDKVKVRVLTMKERRPILGDKFTTRTSQKGTVAYVADET
ELPYDENGITPDVIINSTSIFSRKTISMLIEVILTAAYSAKPYNNKGENR
PVCFPSSNETSIDTYMQFAKQGYEHSNPKLSDEELSDKIFCEKILYDPET
DKPYASKVFFGPIYYLRLRHLTQDKATVRCRGKKTKLIRQANEGRKRGGG
IKFGEMERDCLIAHGAANTITEVLKDSEEDYQDVYVCENCGDIAAQIKGI
NTCLRCSKLNLSPLLTKIDTTHVSKVFLTQMNARGVKVKLDFERRPPSFY
KPLDKVDLKPSFLV
A33R (VACV COP 191)

(SEQ ID NO: 4)

MMTPENDEEQTSVFSATVYGDKIQGKNKRKRVIGLCIRISMVISLLSMIT
MSAFLIVRLNQCMSANEAAITDAAVAVAAASSTHRKVASSTTQYDHKESC
NGLYYQGSCYILHSDYQLFSDAKANCTAESSTLPNKSDVLITWLIDYVED
TWGSDGNPITKTTSDYQDSDVSQEVRKYFCVKTMN
A4BR (VACVgp217)

(SEQ ID NO: 5)

MSRGALIVFEGLDKSGKTTQCMNIMESIPANTIKYLNFPQRSTVTGKMID
DYLTRKKTYNDHIVNLLFCANRWEFASFIQEQLEQGITLIVDRYAFSGVA
YAAAKGASMTLSKSYESGLPKPDLVIFLESGSKEINRNVGEEIYEDVTFQ
QKVLQEYKKMIEEGDIHWQIISSEFEEDVKKELIKNIVIEAIHTVTGPVG
QLWM
A50R (VACVgp219)

(SEQ ID NO: 6)

MTSLREFRKLCCDIYHASGYKEKSKLIRDFITDRDDKYLIIKLLLPGLDD
RIYNMNDKQIIKLYSIIFKQSQEDMLQDLGYGYIGDTIRTFFKENTEIRP
RDKSILTLEDVDSFLTTLSSVTKESHQIKLLTDIASVCTCNDLKCVVMLI
DKDLKIKAGPRYVLNAISPNAYDVFRKSNNLKEIIENSSKQNLDSISISV
MTPINPMLAESCDSVNKAFKKFPSGMFAEVKYDGERVQVHKNNNEFAFFS
RNMKPVLSHKVDYLKEYIPKAFKKATSIVLDSEIVLVDEHNVPLPFGSLG
IHKKKEYKNSNMCLFVEDCLYFDGFDMTDIPLYERRSFLKDVMVEIPNRI
VFSELTNISNESQLTDVLDDALTRKLEGLVLKDINGVYEPGKRRWLKIKR
DYLNEGSMADSADLVVLGAYYGKGAKGGIMAVFLMGCYDDESGKWKTVTK
CSGHDDNTLRVLQDQLTMIKINKDPKKIPEWLVVNKIYIPDFVVEDPKQS
QIWEISGAEFTSSKSHTANGISIRFPRFTRIREDKTWKESTHLNDLVNLT
KS
A57R (VACV gp231)

(SEQ ID NO: 7)

MEREGVDYHYVNREAIWKGIAAGNFLEHTEFLGNIYGTSKTAVNTAAINN
RICVMDLNIDGVRSFKNTYLMPYSVYIRPTSLKMVETKLRCRNTEANDEI
HRRVILAKTDMDEANEAGLFDTIIIEDDVNLAYSKLIQILQDRIRMYFNT
N
C12L (VACV gp018)

(SEQ ID NO: 8)

MDIFKELIVKHPDENVLISPVSILSTLSILNHGAAGSTAEQLSKYIENMN
ENTPDDNNDMDVDIPYCATLATANKIYGSDSIEFHASFLQKIKDDFQTVN
FNNANQTKELINEWVKTMTNGKINSLLTSPLSINTRMTVVSAVHFKAMWK
YPFSKHLTYTDKFYISKNIVTSVDMMVGTENNLQYVHINELFGGFSIIDI
PYEGNSSMVIILPDDIEGIYNIEKNITDEKFKKWCGMLSTKSIDLYMPKF
KVEMTEPYNLVPILENLGLTNIFGYYADFSKMCNETTTVEKFLHTTFIDV
NEEYTEASAVTGVFTINFSMVYRTKVYINHPFMYMIKDTTGRILFIGKYC
YPQ
D1R (VACVgp131)

(SEQ ID NO: 9)

MDANVVSSSTIATYIDALAKNASELEQRSTAYEINNELELVFIKPPLITL
TNVVNISTIQESFIRFTVTNKEGVKIRTKIPLSKVHGLDVKNVQLVDAID
NIVWEKKSLVTENRLHKECLLRLSTEERHIFLDYKKYGSSIRLELVNLIQ
AKTKNFTIDFKLKYFLGSGAQSKSSLLHAINHPKSRPNTSLEIEFTPRDN
EKVPYDELIKELTTLSRHIFMASPENVILSPPINAPIKTFMLPKQDIVGL
DLENLYAVTKTDGIPITIRVTSNGLYCYFTHLGYIIRYPVKRIIDSEVVV
FGEAVKDKNWTVYLIKLIEPVNAINDRLEESKYVESKLVDICDRIVFKSK
KYEGPFTTTSEVVDMLSTYLPKQPEGVILFYSKGPKSNIDFKIKKENTID
QTANVVFRYMSSEPIIFGESSIPYEYKKFSNDKGFPKEYGSGKIVLYNGV
NYLNNIYCLEYINTHNEVGIKSVVVPIKFIAEFLVNGEILKPRIDKTMKY
INSEDYYGNQHNIIVEHLRDQSIKIGDIFNEDKLSDVGHQYANNDKFRLN
PEVSYFTNKRTRGPLGILSNYVKTLLISMYCSKTFLDDSNKRKVLAIDFG
NGADLEKYFYGEIALLVATDPDADAIARGNERYNKLNSGIKTKYYKFDYI
QETIRSDTFVSSVREVFYFGKFNIIDWQFAIHYSFHPRHYATVMNNLSEL
TASGGKVLITTMDGDKLSKLTDKKTFIIHKNLPSSENYMSVEKIADDRIV
VYNPSTMSTPMTEYIIKKNDIVRVFNEYGFVLVDNVDFATIIERSKKFIN
GASTMEDRPSTKNFFELNRGAIKCEGLDVEDLLSYYVVYVFSKR
D5R (VACVgp138)

(SEQ ID NO: 10)

MDAAIRGNDVIFVLKTIGVPSACRQNEDPRFVEAFKCDELKRYIDNNPEC
TLFESLRDEEAYSIVRIFMDVDLDACLDEIDYLTAIQDFIIEVSNCVARF
AFTECGAIHENVIKSMRSNFSLTKSTNRDKTSFHIIFLDTYTTMDTLIAM
KRTLLELSRSSENPLTRSIDTAVYRRKTTLRVVGTRKNPNCDTIHVMQPP
HDNIEDYLFTYVDMNNNSYYFSLQRRLEDLVPDKLWEPGFISFEDAIKRV
SKIFINSIINFNDLDENNFTTVPLVIDYVTPCALCKKRSHKHPHQLSLEN
GAIRIYKTGNPHSCKVKIVPLDGNKLFNIAQRILDTNSVLLTERGDYIVW
INNSWKFNSEEPLITKLILSIRHQLPKEYSSELLCPRKRKTVEANIRDML
VDSVETDTYPDKLPFKNGVLDLVDGMFYSGDDAKKYTCTVSTGFKFDDTK
FVEDSPEMEELMNIINDIQPLTDENKKNRELYEKTLSSCLCGATKGCLTF
FFGETATGKSTTKRLLKSAIGDLFVETGQTILTDVLDKGPNPFIANMHLK
RSVFCSELPDFACSGSKKIRSDNIKKLTEPCVIGRPCFSNKINNRNHATI
IIDTNYKPVFDRIDNALMRRIAVVRFRTHFSQPSGREAAENNDAYDKVKL
LDEGLDGKIQNNRYRFAFLYLLVKWYKKYHVPIMKLYPTPEEIPDFAFYL
KIGTLLVSSSVKHIPLMTDLSKKGYILYDNVYTLPLTTFQQKISKYFNSR
LFGHDIESFINRHKKFANVSDEYLQYIFIEDISSP
E3L (VACV gp075)

(SEQ ID NO: 11)

MSKIYIDERSDAEIVCAAIKNIGIEGATAAQLTRQLNMEKREVNKALYDL
QRSAMVYSSDDIPPRWFMTTEADKPDADAMADVIIDDVSREKSMREDHKS
FDDVIPAKKIIDWKDANPVTIINEYGQITKRDWSFRIESVGPSNSPTFYA
CVDIDGRVFDKADGKSKRDAKNNAAKLAVDKLLGYVIIRF
F3 (VACV gp067)

(SEQ ID NO: 12)

MVIGLVIFVSVAAAIVGVLSNVLDMFMYVEENNEEDARIKEEQELLLLY
F12L (VACV gp063)

(SEQ ID NO: 13)

MLNRVQILMKTANNYETIEILRNYLRLYIILARNEEGHGILIYDDNIDSI
MSMMNITRLEVIGLTTHCTKLRSSPPIPMSRLFMDEIDHESYYSPKTSDY
PLIDIIRKRSHEQGDIALALEQYGIENTDSISEINEWLSSKGLACYRFVK
FNDYRKQMYRKFSRCTIVDSMIIGHIGHHYIWKNLETYTRPEIDVLPFDI
KYISRDELWVRISSSLDQTHIKTIAVSVYGAITDNGPIPYMISTYPGNTF
VNFNSVKNLILNFLDWIKDIMTSTRTIILVGYMSNLFDIPLLTVYWPNNC
GWKIYNNTLISSDGARVIWMDAYKFSCGLSLQDYCYHWGSKPESRPFDLI
KKSDAKRNSKSLVKESMASLKSLYEAFETQSGALEVLMSPCRMFSFSRIE
DMFLTSVINRVSENTGMGMYYPTNDIPSLFIESSICLDYIIVNNQESNKY
RIKSVLDIISSKQYPAGRPNYVKNGTKGKLYIALCKVTVPTNDHIPVVYH
DDDNTTTFITVLTSVDIETAIRAGYSIVELGALQWDNNIPELKNGLLDSI
KMIYDLNAVTTNNLLEQLIENINFNNSSIISLFYTFAISYCRAFIYSIME
TIDPVYISQFSYKELYVSSSYKDINESMSQMVKL
I3L (VACVgp093)

(SEQ ID NO: 14)

MSKVIKKRVETSPRPTASSDSLQTCAGVIEYAKSISKSNAKCIEYVTLNA
SQYANCSSISIKLTDSLSSQMTSTFIMLEGETKLYKNKSKQDRSDGYFLK
IKVTAASPMLYQLLEAVYGNIKHKERIPNSLHSLSVETTTEKTFKDESIF
INKLNGAMVEYVSAGESSILRSIEGELESLSKRERQLAKAIITPIVFYRS
GTETKITFALKKLIIDREVVANVIGLSGDSERVSMTENVEEDLARNLGLV
DIDDEYDEDSDKEKPIFNV
IL-18bp (VACWR013)

(SEQ ID NO: 15)

MRILFLIAFMYGCVHPYVNADEIKCPNLNIVTSSGEFRCTGCVKFMPNFS
YMYWLAKDMRSDEDAKFIEHLGEGIKEDETVSTIDGRIVTLQKVLHVTDT
NKFDNYRFTCVLTTIDGVSKKNIWLK
IL-18bp-like protein

(amino acids 59-126; SEQ ID NO: 16)

RSDEDTKFIEHLGDGIKEDETVRTTDSGITTLRKVLHVTDTNKFAHYRF
TCVLTTIDGVSKKNIWLK
L1R (VACY COP 107)

(SEQ ID NO: 17)

MGAAASIQTTVNTLSERISSKLEQEANASAQTKCDIEIGNFYIRQNHGCN
LTVKNMCSADADAQLDAVLSAATETYSGLTPEQKAYVPAMFTAALNIQTS
VNTVVRDFENYVKQTCNSSAVVDNKLKIQNVIIDECYGAPGSPTNLEFIN
TGSSKGNCAIKALMQLTTKATTQIAPRQVAGTGVQFYMIVIGVIILAALF
MYYAKRMLFTSTNDKIKLILANKENVHWTTYMDTFFRTSPMVIATTTDMQ
N
M2L (VACV gp038)

(SEQ ID NO: 18)

MVYKLVLLFCIASLGYSVEYKNTICPPRQDYRYWYFAAELTIGVNYDINS
TIIGECHMSESYIDRNANIVLTGYGLEINMTIMDTDQRFVAAAEGVGKDN
KLSVLLFTTQRLDKVHHNISVTITCMEMNCGTTKYDSDLPESIHKSSSCD
ITINGSCVTCVNLETDPTKINPHYLHPKDKYHYHNSEYGMRGSYGVTFID
ELNQCLLDIKELSYDICYRE

In general, polypeptides (including fusion proteins) and polynucleotides as described herein are isolated. An “isolated” polypeptide or polynucleotide is one that is removed from its original environment. For example, a naturally occurring protein is isolated if it is separated from some or all of the coexisting materials in the natural system. An isolated vaccinia polypeptide of the invention is one that has been isolated, produced or synthesized such that it is separate from a complete, native vaccinia virus, although the isolated polypeptide may subsequently be introduced into a recombinant vaccinia or other virus. A recombinant vaccinia virus that comprises an isolated polypeptide or polynucleotide of the invention is an example of subject matter provided by the invention. Preferably, such isolated polypeptides are at least about 90% pure, more preferably at least about 95% pure and most preferably at least about 99% pure. A polynucleotide is considered to be isolated if, for example, it is cloned into a vector that is not part of the natural environment.

The polypeptide can be isolated from its naturally occurring form, produced by recombinant means or synthesized chemically. Recombinant polypeptides encoded by DNA sequences described herein can be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably the host cells employed are E. coli, yeast or a mammalian cell line such as Cos or CHO. Supernatants from the soluble host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Fragments and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, wherein amino acids are sequentially added to a growing amino acid chain (Merrifield, 1963, J. Am. Chem. Soc. 85:2146-2149). Equipment for automated synthesis of polypeptides is commercially available from suppliers such as Perlin Elmer/Applied BioSystems Division (Foster City, Calif.), and may be operated according to the manufacturer's instructions.

Variants of the polypeptide for use in accordance with the invention can have one or more amino acid substitutions, deletions, additions and/or insertions in the amino acid sequence indicated that result in a polypeptide that retains the ability to elicit an immune response to vaccinia or vaccinia-infected cells. Such variants may generally be identified by modifying one of the polypeptide sequences described herein and evaluating the reactivity of the modified polypeptide using a known assay such as a T cell assay described herein. Polypeptide variants preferably exhibit at least about 70%, more preferably at least about 90%, and most preferably at least about 95% identity to the identified polypeptides. These amino acid substitutions include, but are not necessarily limited to, amino acid substitutions known in the art as “conservative”.

A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

One can readily confirm the suitability of a particular variant by assaying the ability of the variant polypeptide to elicit an immune response. The ability of the variant to elicit an immune response can be compared to the response elicited by the parent polypeptide assayed under identical circumstances. One example of an immune response is a cellular immune response. The assaying can comprise performing an assay that measures T cell stimulation or activation. Examples of T cells include CD4 and CD8 T cells.

One example of a T cell stimulation assay is a cytotoxicity assay, such as that described in Koelle, D M et al., Human Immunol. 1997, 53; 195-205. In one example, the cytotoxicity assay comprises contacting a cell that presents the antigenic viral peptide in the context of the appropriate HLA molecule with a T cell, and detecting the ability of the T cell to kill the antigen presenting cell. Cell killing can be detected by measuring the release of radioactive ⁵¹Cr from the antigen presenting cell. Release of ⁵¹Cr into the medium from the antigen presenting cell is indicative of cell killing. An exemplary criterion for increased killing is a statistically significant increase in counts per minute (cprn) based on counting of ⁵¹Cr radiation in media collected from antigen presenting cells admixed with T cells as compared to control media collected from antigen presenting cells admixed with media.

### Fusion Proteins

The polypeptide can be a fusion protein. In one embodiment, the fusion protein is soluble. A soluble fusion protein of the invention can be suitable for injection into a subject and for eliciting an immune response. Within certain embodiments, a polypeptide can be a fusion protein that comprises multiple polypeptides as described herein, or that comprises at least one polypeptide as described herein and an unrelated sequence. In one example, the fusion protein comprises a vaccinia epitope described herein (with or without flanking adjacent native sequence) fused with non-native sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985, Gene 40:39-46; Murphy et al., 1986, Proc. Nad. Acad. Sci. USA 83:8258-8262; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptides. Similarly, stop codons required to end translation and transcription termination signals are present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al., 1997, New Engl. J. Med., 336:86-9).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenza virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In some embodiments, it may be desirable to couple a therapeutic agent and a polypeptide of the invention, or to couple more than one polypeptide of the invention. For example, more than one agent or polypeptide may be coupled directly to a first polypeptide of the invention, or linkers that provide multiple sites for attachment can be used. Alternatively, a carrier can be used. Some molecules are particularly suitable for intercellular trafficking and protein delivery, including, but not limited to, VP22 (Elliott and O'Hare, 1997, Cell 88:223-233; see also Kim et al., 1997, J. Immunol. 159:1666-1668; Rojas et al., 1998, Nature Biotechnology 16:370; Kato et al., 1998, FEBS Lett. 427(2):203-208; Vives et al., 1997, J. Biol. Chem. 272(25):16010-7; Nagahara et al., 1998, Nature Med. 4(12):1449-1452).

A carrier may bear the agents or polypeptides in a variety of ways, including covalent bonding either directly or via a linker group. Suitable carriers include proteins such as albumins (e.g., U.S. Pat. No. 4,507,234, to Iato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088).

### Polynucleotides, Vectors, Host Cells and Recombinant Viruses

The invention provides polynucleotides that encode one or more polypeptides of the invention. The complete genome sequence of vaccinia, Copenhagen strain, has been deposited with Genbank, Accession No. NC_{—}001559. The polynucleotide can be included in a vector. The vector can further comprise an expression control sequence operably linked to the polynucleotide of the invention. In some embodiments, the vector includes one or more polynucleotides encoding other molecules of interest. In one embodiment, the polynucleotide of the invention and an additional polynucleotide can be linked so as to encode a fusion protein.

Within certain embodiments, polynucleotides may be formulated so to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, vaccinia or a pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

The invention also provides a host cell transformed with a vector of the invention. The transformed host cell can be used in a method of producing a polypeptide of the invention. The method comprises culturing the host cell and recovering the polypeptide so produced. The recovered polypeptide can be purified from culture supernatant.

Vectors of the invention can be used to genetically modify a cell, either in vivo, ex tivo or in vitro. Several ways of genetically modifying cells are known, including transduction or infection with a viral vector either directly or via a retroviral producer cell, calcium phosphate precipitation, fusion of the recipient cells with bacterial protoplasts containing the DNA, treatment of the recipient cells with liposomes or microspheres containing the DNA, DEAE dextran, receptor-mediated endocytosis, electroporation, micro-injection, and many other techniques known to those of still. See, e.g., Sambrook et al. Molecular Cloning—A Laboratory Manual (2nd ed.) 1-3, 1989; and Current Protocols in Molecular Biology, F. M. Ausubel et al., eds., Greene Publishing Associates, Inc. and John Wiley & Sons, Inc., (1994 Supplement).

Examples of viral vectors include, but are not limited to retroviral vectors based on, e.g., HIV, SIV, and murine retroviruses, gibbon ape leukemia virus and other viruses such as adeno-associated viruses (AAVs) and adenoviruses. (Miller et al. 1990, Mol. Cell Biol. 10:4239; J. Kolberg 1992, NIH Res. 4:43, and Cornetta et al. 1991, Hum. Gene Ther. 2:215). Widely used retrovital vectors include those based upon murine leukemia virus (MuLV), gibbon ape leukemia virus (GaLV), ecottopic retroviruses, simian immunodeficiency virus (SIV), human immunodeficiency virus (HIV), and combinations. See, e.g. Buchscher et al. 1992, J. Virol. 66(5):2731-2739; Johann et al. 1992, J. Virol. 66(5):1635-1640; Sommerfelt et al. 1990, Virol. 176:58-59; Wilson et al. 1989, J. Virol. 63:2374-2378; Miller et al. 1991, J. Virol. 65:2220-2224, and Rosenberg and Fauci 1993 in Fundamental Immunology, Third Edition, W. E. Paul (ed.) Raven Press, Ltd., New York and the references therein; Miller et al. 1990, Mol. Cell. Biol. 10:4239; R. Kolberg 1992, J. NIH Res. 4:43; and Cornetta et al. 1991, Hum. Gene Ther. 2:215.

In vitro amplification techniques suitable for amplifying sequences to be subcloned into an expression vector are known. Examples of such in vitro amplification methods, including the polymerase chain reaction (PCR), ligase chain reaction (LCR), Qβ-replicase amplification and other RNA polymerase mediated techniques (e.g., NASBA), are found in Sambrook et al. 1989, Molecular Cloning—A Laboratory Manual (2nd Ed) 1-3; and U.S. Pat. No. 4,683,202; PCR Protocols A Guide to Methods and Applications (Innis et al. eds.) Academic Press Inc. San Diego, Calif. 1990. Improved methods of cloning in vitro amplified nucleic acids are described in U.S. Pat. No. 5,426,039.

The invention additionally provides a recombinant microorganism genetically modified to express a polynucleotide of the invention. The recombinant microorganism can be useful as a vaccine, and can be prepared using techniques known in the art for the preparation of live attenuated vaccines. Examples of microorganisms for use as live vaccines include, but are not limited to, viruses and bacteria. In a preferred embodiment, the recombinant microorganism is a virus. Examples of suitable viruses include, but are not limited to, vaccinia virus and other poxviruses.

### Compositions

The invention provides compositions that are useful for treating and preventing vaccinia infection. The compositions can be used to inhibit viral replication and to kill virally-infected cells. In one embodiment, the composition is a pharmaceutical composition. The composition can comprise a therapeutically or prophylactically effective amount of a polypeptide, polynucleotide, recombinant virus, APC or immune cell of the invention. An effective amount is an amount sufficient to elicit or augment an immune response, e.g., by activating T cells. One measure of the activation of T cells is a cytotoxicity assay, as described in D. M. Koelle et al., 1997, Human Immunol. 53:195-205. In some embodiments, the composition is a vaccine.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other viral antigens may be present, either incorporated into a fusion polypeptide or as a separate compound, within the composition or vaccine.

A pharmaceutical composition or vaccine may contain DNA encoding one or more of the polypeptides of the invention, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. My Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91102805; Berkner, 1988, Biotechniques 6:616-627; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cit. Res. 73:1202-1207. Techniques for incorporating DNA into such expression systems are well known to those of ordinary skill in the art. The DNA may also be “naked,” as described, for example, in Ulmer et al., 1993, Science 259:1745-1749 and reviewed by Cohen, 1993, Science 259:1691-1692. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of adjuvants may be employed in the vaccines of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; monophosphoryl lipid A and quil A. Cytokines, such as GM CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytolines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPLTM adjuvants are available from Corixa Corporation (see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets vaccinia-infected cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have antiviral effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic immunity (see Timmerman and Levy, Ann. Rev. Med. 50:507-529,1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural liller cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al., 1998, Nature Med. 4:594-600).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86).

APCs may generally be transfected with a polynucleotide encoding a polypeptide (or portion or other variant thereof such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in tivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The compositions are administered in any suitable manner, often with pharmaceutically acceptable carriers. Suitable methods of administering cells in the context of the present invention to a patient are available, and, although more than one route can be used to administer a particular cell composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as vaccinia infection, the physician needs to evaluate the production of an immune response against the virus, progression of the disease, and any treatment-related toxicity.

For example, a vaccine or other composition containing a subunit vaccinia protein can include 1-10,000 micrograms of vaccinia protein per dose. In a preferred embodiment, 10-1000 micrograms of vaccinia protein is included in each dose in a more preferred embodiment 10-100 micrograms of vaccinia protein dose. Preferably, a dosage is selected such that a single dose will suffice or, alternatively, several doses are administered over the course of several months. For compositions containing vaccinia polynucleotides or peptides, similar quantities are administered per dose.

In one embodiment, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an antiviral immune response, and is at least 10-50% above the basal (i.e., untreated) level. Such vaccines should also be capable of causing an immune response that leads to an improved clinical outcome in vaccinated patients as compared to non-vaccinated patients. In general, for pharmaceutical compositions and vaccines comprising one or more polypeptides, the amount of each polypeptide present in a dose ranges from about 0.1 μg to about 5 mg per kg of host. Preferably, the amount ranges from about 10 to about 1000 μg per dose. Suitable volumes for administration will vary with the size, age and immune status of the patient, but will typically range from about 0.1 mL to about 5 mL, with volumes less than about 1 mL being most common.

Compositions comprising immune cells are preferably prepared from immune cells obtained from the subject to whom the composition will be administered. Alternatively, the immune cells can be prepared from an HLA-compatible donor. The immune cells are obtained from the subject or donor using conventional techniques known in the art, exposed to APCs modified to present an epitope of the invention, expanded ex vivo, and administered to the subject. Protocols for ex vivo therapy are described in Rosenberg et al., 1990, New England J. Med. 9:570-578. In addition, compositions can comprise APCs modified to present an epitope of the invention.

Immune cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to enrich and rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., 1997, Immunological Reviews 157:177).

Administration by many of the routes of administration described herein or otherwise known in the art may be accomplished simply by direct administration using a needle, catheter or related device, at a single time point or at multiple time points.

### In Vivo Testing of Identified Antigens

Conventional techniques can be used to confirm the in vivo efficacy of the identified vaccinia antigens. For example, one technique makes use of a mouse challenge model. Those skilled in the art, however, will appreciate that these methods are routine, and that other models can be used. There is a monkey model for virulent variola infection, for example, Jahtling P B et al., “Exploring the potential of variola virus infection of cynomolgus macaques as a model for human smallpox” Proc Natl Acad Sci USA. 2004 Oct 19; 101 (42):15196-200. Given the dangers inherent in working with variola, however, those skilled in the art are more likely to rely on inferential data derived from in vitro studies and experience with other vaccines and observations of protective immunity.

Once a compound or composition to be tested has been prepared, the mouse or other subject is immunized with a series of injections. For example up to 10 injections can be administered over the course of several months, typically with one to 4 weeks elapsing between doses. Following the last injection of the series, the subject is challenged with a dose of virus established to be a uniformly lethal dose. A control group receives placebo, while the experimental group is actively vaccinated. Alternatively, a study can be designed using sublethal doses. Optionally, a dose-response study can be included. The end points to be measured in this study include death and severe neurological impairment, as evidenced, for example, by spinal cord gait. Survivors can also be sacrificed for quantitative viral cultures of key organs including spinal cord, brain, and the site of injection. The quantity of virus present in ground up tissue samples can be measured. Compositions can also be tested in previously infected animals for reduction in recurrence to confirm efficacy as a therapeutic vaccine.

Efficacy can be determined by calculating the IC₅₀, which indicates the micrograms of vaccine per kilogram body weight required for protection of 50% of subjects from death. The IC₅₀ will depend on the challenge dose employed. In addition, one can calculate the LD₅₀, indicating how many infectious units are required to kill one half of the subjects receiving a particular dose of vaccine. Determination of the post mortem viral titer provides confirmation that viral replication was limited by the immune system.

### Methods of Treatment and Prevention

The invention provides a method for treatment and/or prevention of poxvirus infection in a subject. The method comprises administering to the subject a composition of the invention. The composition can be used as a therapeutic or prophylactic vaccine. In one embodiment, the poxvirus is smallpox. Alternatively, the poxvirus is monkeypox or another orthopox virus. The invention additionally provides a method for inhibiting viral replication, for killing virally-infected cells, for increasing secretion of lymphokines having antiviral and/or immunomodulatory activity, and for enhancing production of virus-specific antibodies. The method comprises contacting an infected cell with an immune cell directed against an antigen of the invention, for example, as described in the Examples presented herein. The contacting can be performed in vitro or in vivo. In a preferred embodiment, the immune cell is a T cell. T cells include CD4 and CD8 T cells. Compositions of the invention can also be used as a tolerizing agent against immunopathologic disease.

In addition, the invention provides a method of producing immune cells directed against poxvirus. The method comprises contacting an immune cell with a polypeptide of the invention. The immune cell can be contacted with the polypeptide via an antigen-presenting cell, wherein the antigen-presenting cell is modified to present an antigen included in a polypeptide of the invention. Preferably, the antigen-presenting cell is a dendritic cell. The cell can be modified by, for example, peptide loading or genetic modification with a nucleic acid sequence encoding the polypeptide. In one embodiment, the immune cell is a T cell. T cells include CD4 and CD8 T cells. Also provided are immune cells produced by the method. The immune cells can be used to inhibit viral replication, to kill virally-infected cells, in vitro or in vivo, to increase secretion of lymphokines having antiviral and/or immunomodulatory activity, to enhance production of virus-specific antibodies, or in the treatment or prevention of viral infection in a subject.

### Methods of Detecting Infection

The invention also provides methods and kits for detecting poxvirus infection in a subject, and a method for detecting whether a candidate vaccine to prevent variola has elicited a T-cell immune response. In one embodiment, the diagnostic assay can be used to identify the immunological responsiveness of a patient suspected of having a poxvirus infection and to predict responsiveness of a subject to a particular course of therapy. The assay comprises exposing T cells of a subject to an antigen of the invention, in the context of an appropriate APC, and testing for immunoreactivity by, for example, measuring IFNγ, proliferation or cytotoxicity.

In one embodiment, the invention provides a method for detecting poxvirus infection in a subject, wherein the method comprises contacting a biological sample obtained from the subject with a molecule of the invention (e.g., polypeptide, polynucleotide, antibody); and detecting the presence of a binding agent that binds to the molecule in the sample, thereby detecting poxvirus infection in the biological sample. Optionally, the molecule to be detected is labeled with a detectable marker. Examples of biological samples include, but are not limited to, whole blood, sputum, serum, plasma, saliva, cerebrospinal fluid and urine. In one embodiment, the kit comprises a polypeptide of the invention in combination with a detectable marker. In another embodiment, the kit comprises a monoclonal antibody or a polyclonal antibody that binds with a polypeptide of the invention.

### EXAMPLES

The following examples are presented to illustrate the present invention and to assist one of ordinary skill in making and using the same. The examples are not intended in any way to otherwise limit the scope of the invention.

### Example 1

### Diversity in the Acute CD8 T Cell Response to Vaccinia Virus in Humans

This example examines the fine specificity of cloned and bulk human vaccinia-specific CD8 CTL by expressing polypeptide fragments from a library of vaccinia genomic DNA. This epitope discovery method emphasizes virus-specific biological activity, as the responder cells are all reactive with whole vaccinia virus. Sixteen novel epitopes, restricted by several HLA A and B alleles, were defined to the nonamer peptide level in diverse vaccinia open reading frames. An additional seven epitopes were mapped to short regions of vaccinia proteins. Targets of the CD8 response included proteins assigned to structural, enzymatic, transcription factor, and immune evasion functions, and included members of all viral kinetic classes. Most epitopes were conserved in other orthopoxviruses. Responses to at least 18 epitopes were detected within a single blood sample, revealing a surprising degree of diversity. These epitopes will be useful in natural history studies of CD8 responses to vaccinia, a nonpersisting virus with long-term memory, and in the design and evaluation of attenuated and replication-incompetent vaccinia strains for variola and monkeypox prevention and for the delivery of heterologous Ags.

Various references are cited throughout this example by numerals in parentheses. The corresponding citations can be found in a numbered list at the end of this example.

### Materials and Methods

### Subjects and Specimens

Eight adult subjects (Table I) receiving scarification with Dryvax smallpox vaccine for occupational health were consented after approval by the Institutional Review Board. Five had received one previous vaccination, ranging from 32 to 43 years before recent immunization, while one had received two previous vaccinations 28 and 52 years prior to reimmunization and two younger individuals were primary vaccinees. PBMC from peripheral blood obtained by phlebotomy into sodium heparin-anticoagulated syringes at weeks 2, 4, and 6 after vaccination were enriched by Ficoll centrifugation from peripheral blood and cryopreserved. No relationship between time after vaccination, or between primary vs. revaccination status, and the yield of mononuclear cells per volume of blood was noted. HLA typing was done at the Puget Sound Blood Center (Seattle, Wash.).

### Cell and Viral Culture

PBMC were seeded at 10⁶/ml in 2 ml of T cell medium (TCM) in 24-well plates (14). Live vaccinia at a multiplicity of infection (MOI) of 10 was added to restimulate lymphocytes (15). IL-2 (Hemagen) was begun on day 5 (32 U/ml). Cultures were split as needed, fed periodically with IL-2-TCM, and CTL assays done on days 12-14. CDS magnetic-positive selection (Miltenyi Biotec or StemCell Technologies), typically yielding >95% CD8+ cells, was followed by functional assays (below), cloning with PHA as mitogen, or bulk T cell expansion with anti-CD3 as mitogen (14). Clones were screened (day 14) by CTL assay. Positive clones were expanded (14) to >10⁸ cells and used, or frozen, at the end of an expansion cycle. EBV-transformed B-lymphocyte continuous lines (LCL) were derived from PBMC (16). Vaccinia strain New York City Board of Health (NYCBH; National Institutes of Health Aids Research and Reference Reagent Program, Germantown, MD) was raised and titered in BSC-40 cells (16). Cos-7 and BSC-40 were cultured in DMEM with 10% FCS.

### Lymphocyte Functional Assays

⁵¹Cr CTL assays used autologous mock- and vaccinia-infected (MOI 10, 18 h) LCL, or peptide-pulsed LCL (90 min, 37° C.) at 2×10³/well as targets (16). Candidate clones were screened singly or in duplicate. Clones with >20% specific release for vaccinia-infected LCL and >10% for uninfected targets were expanded. Established clones, and bulk cultures, were triplicate tested at 20 effectors/target. Percent-specific release was calculated (16); spontaneous release (16) was usually <25%. To assign restricting HLA class I alleles to CTL clones, panels of allogeneic LCL matched at one or more HLA class I alleles were used as APC with and without vaccinia infection. Patterns of results were analyzed for informative, nonambiguous restriction (14, 17) (see Results).

T cell activation was detected by IFN-γ ELISA of culture supernatants (17). Exponential standard curves were used to convert OD450 values to cytokine concentrations and the level of IFN-γ secreted by nonstimulated T cells subtracted to give specific secretion. For intracellular cytokine cytometry (ICC) (18), peptides (1 μM) were added to 3-5×10⁵ bulk-cultured T cells in 500 μl of TCM for 15 h. A total of 1×10⁵ autologous LCL were added as APC. Anti-CD28 and anti-CD49d, and brefeldin A, were added at 0 and 1 h, respectively (18). Each specimen was stained with anti-CD8-PE-Cyanin 5 (Cy5) or -FITC, permeabilized, and then split for staining with control mAb-PE or anti-IFN-γ-PE. Controls were DMSO (1%) and PMA/ionomycin (18).

### Flow Cytometry

Bulk cultures were stained with anti-TCRαβ-FITC (3D Biosciences), anti-CD4-PE, and anti-CD8+-PE-Cy5 (Caltag Laboratories). PE-labeled tetrameric complexes of HLA B*0801 and peptide A50R 395-403 (WLKIKRDYL; SEQ ID NO: 27) supplied by the National Institutes of Health Tetramer Program (Atlanta, Ga.) was used at 0.1 μl/5×10⁵ cells in 75 μl of TCM, 60 min, room temperature, followed by anti-CD8-PE-Cy5 for 30 min, 4° C. Clones were stained with anti-TCRαβ and anti-CD8. HLA expression by 48-h transfected Cos-7 was measured by staining HLA-specific mAb (One Lambda; unlabeled, or biotin- or FITC-conjugated) and goat anti-mouse PE or streptavidin-PE (BD Biosciences). ICC data are reported as the percentage of CD8+ lymphocytes that stain positive for IFN-γ (see Results). Data collected on FACScan (BD Biosciences) were analyzed with WinMDI 2.8 (http://facs.scripps.edu/software.html).

### Caccinia Genomic Library

BSC-40 cells at 90% confluent were infected 48 h with vaccinia NYCBH, MOI 10. Nuclear DNA was reduced by lysing cells (450 cm²) with 1% Nonidet P-40 (17), centrifugation (400×g, 15 min), and retention of the supernatant. The cytoplasmic fraction was extracted with chloroform-phenol and DNA precipitated with ethanol (17). Vaccinia DNA was digested with DNase I (New England Biolabs) with optimized MnCl2 concentration, temperature, and enzyme/substrate ratio to generate DNA fragments in the 0.1-2 kB range. DNA was purified from the excised agarose gel zone corresponding to 300-500 bp (Qiaquick). Termini were blunt-ended with T4 DNA polymerase and dNTPs. The gel-purified purified blunt-end fragments were ligated to a dsDNA adaptor with a 5′ GA overhang: 5′-GAGGGTCCGACAGC (SEQ ID NO: 19; single-stranded overhangs are underlined). Unincorporated linkers were removed by gel purification. The library vector backbone (pEGFP-C1; BD Clontech) was XhoI-digested, partially filled in with dTTp and dCTP, and gel-purified to give TC overhangs complementary to the vaccinia fragments. After ligation and purification of DNA by organic extraction/ethanol precipitation, libraries were created by electroporation (BTX) of Escherichia coli DH10B (Invitrogen Life Technologies). Libraries were plated on 10 150-mm diameter kanamycin-LB plates. Bacteria rinsed from the primary growth plates with 10 ml of broth were frozen in aliquots for glycerol stocks, which were titered on kanamycin plates. 96-well deep-dish plates (n=5) were seeded at 40 colonies/well. Resultant plasmid DNA for transfection was prepared (14) with an average yield of 5 μg/well. This yielded a library of 1.9×10⁴ independent vaccinia DNA fragments at a complexity of 40/well. Pools were diluted to an average of 50 ng/μl DNA for screening. Forty single colonies derived from retransformation of selected pools were sequenced to check library insert identity and heterogeneity.

The purity of the vaccinia genomic DNA used for library construction was estimated by restriction endonuclease digestion/agarose electrophoresis. Discrete bands were observed, consistent with reduction of cellular DNA. The primary library was estimated, from counting primary growth plates, to contain 3.0×10⁴ unique kanamycin-resistant colonies. Sequencing of 40 random colonies showed that 90% contained single independent vaccinia DNA inserts, averaging 300-bp long. High diversity was also observed. The quality of the library 96-well miniprep DNA (14), derived from either pools or single bacterial clones, was verified by transfecting Cos-7 cells and observing enhanced GFP (eGFP) live-cell fluorescence in >50% of cells for most DNA preparations.

HLA cDNA Expression Plasmids

HLA A*0101, A*0201, and B*4403 cDNAs in pcDNA3.0 (Invitrogen Life Technologies) have been described (19, 20). HLA B*0801 cDNA in pcDNA 3.0 was obtained from Dr. J. Pei (Fred Hutchinson Cancer Research Center, Seattle, Wash.). For other alleles, RNA was isolated from subjects' LCL (RNAeasy; Qiagen) and first strand cDNA synthesis primed with oligo(dT) (Superscript II; Invitrogen Life Technologies). cDNA template was PCR-amplified (pfu; Invitrogen Life Technologies). HLA A*2301 and A*2902 primers were GGCGCTAGCATGGCCGTCATGGCG (SEQ ID NO:20) and GGCCTCGAGTCACACTTTACAAGCTGTGAGAGAC (SEQ ID NO:21; NheI and XhoI sites underlined). PCR products were digested, gel-purified, and directionally ligated into similarly digested pcDNA3.1 (Invitrogen Life Technologies). Low-endotoxin plasmid DNA was prepared (Qiagen) after sequence verification.

### Epitope Discovery

Details and examples have been published (14, 17). Briefly, functional HLA expression and restriction were confirmed by transfection of Cos-7 cells, plated the day before at 9×10³/well in 96-well flats, with HLA cDNA (50 ng/well) using Fugene 6 (Roche) or Lipofectamine (Invitrogen Life Technologies), followed the next day by vaccinia infection (MOI 2-10). One day later, 5×10⁴ cloned CD8 CTL were added in 130 μl of LCL media (16) with 2 U/ml IL-2. As controls, autologous or HLA-mismatched LCL were mock- or vaccinia-infected overnight at MOI 10 and cocultured (2.5×10⁴ LCL and 5-10×10⁴ CD8 CTL) in 96-well U plates for 24 h. Twenty-four hour supernatants were assayed for IFN-γ. If HLA transfection plus infection lead to high IFN-γ release, as described (17), HLA expression was functionally adequate for library screening.

Cos-7 were transfected with 50 ng of HLA cDNA and 150 ng of library pool DNA/well. We screened 384 library pools in duplicate, the equivalent of 1.5×10⁴ discrete vaccinia genomic fragments. T cells were added 24-48 h later and IFN-γ was measured after an additional day. If multiple positive pools were detected, up to five were analyzed. Positive plasmid pools were broken down by retransformation and selection of 96 single daughter bacterial colonies per positive pool, screened as plasmid DNA in a secondary cotransfection assay. Single, biologically active plasmids were sequenced (17).

Candidate peptides were selected by bioinformatics (14). Briefly, if more than one active plasmid was sequenced, overlapping insert sequences were assembled into a contig (DNASTAR) after trimming. The overlap (or single) region was searched with a basic local alignment search tool (www.poxvitus.org/; Ref. 21). Typically, the vaccinia insert was within a documented/predicted vaccinia ORF and in-frame with eGFP. Some exceptions are discussed in Results. Predicted amino acid sequences in the antigenic fragments were submitted to HLA epitope prediction algorithms (22, 23) and high-scoring peptides (Synpep) dissolved in DMSO. Orthopoxvirus genomes (21, 24) were searched for the presence and sequence of homologous ORFs, antigenic fragments, and peptide epitopes. Alphanumeric ORF nomenclature based on vaccinia Copenhagen HindIII digests, and systematic names, are used (21, 25).

### High-Throughput Epitole Discovery

Peptide epitopes recognized by bulk vaccinia-specific T cells were also identified using a parallel processing variant method. Cos-7 (384 wells) were transfected in duplicate with cDNA encoding one of the subjects' HLA class I A or B alleles, plus the library. Bulk CD8 CTL (10⁵/well) were substituted for cloned CTL as responders. Single active plasmids were sequenced and contigs assembled and analyzed as above. Candidate peptides were tested by loading (0.01-10 μM) onto autologous LCL (2×10⁵ cells, 200 μl of LCL medium, 90 min, 37° C.). After washing, stimulators were plated in duplicate or triplicate with 1×10⁵ bulk CTL responders in 130 μl of TCM with 2 U/ml IL-2 in 96-well U-bottom plates, and T cell activation detected by IFN-γ ELISA in 24-h supernatants. Specific responses at 1 μM or lower were considered positive. As an alternative, bulk CTL were tested with synthetic peptides (1 μM) by IFN-γ ICC as detailed above.

### Results

### Detection and Cloning of Vaccinia-Specific CD8 T Cells

### Bulk CTL.

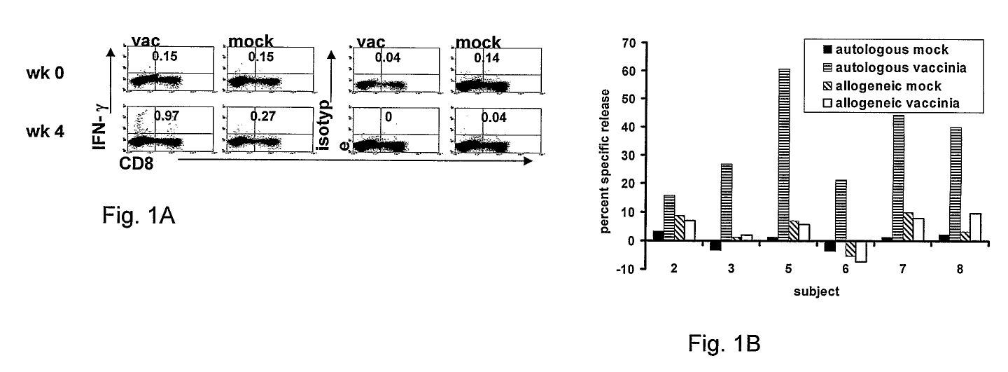

Vaccinia-specific CD8 T cells were initially detected by IFN-γ ICC using whole PBMC responders and live vaccinia stimulation. Specific signals in the range of 1.0% of CD8+ lymphocytes were detected 2-6 wk after Dryvax, but not in vaccinia-naive subjects (FIG. 1, representative subject). To enrich vaccinia-specific CD8 T cells, PBMC from eight subjects (Table I), obtained 2-6 wk after intradermal vaccination, were restimulated once in vitro. Vaccinia-specific, self-restricted cytotoxicity was detected, as defined in Materials and Methods, in each subject except subject 1. These cultures were predominantly CD8+, CD4⁻, and >95% TCRαβ⁺. CD8+ cells were purified from six cultures. For each, strong virus- and self-restricted CTL activity was detected (FIG. 1).

TABLE I
Subjects' vaccination status and time after vaccination for
PBMC specimens.

| | | number and timing of | | |
|---|---|---|---|---|
| subject | vaccination | previous vaccinations¹ | | time point² |
| 1 | re-vaccination | 1 | (43) | 2 |
| 2 | primary | 0 | | 6 |
| 3 | re-vaccination | 1 | (34) | 2 |
| 4 | re-vaccination | 1 | (32) | 2 |
| 5 | re-vaccination | 2 | (52, 28) | 4 |
| 6 | re-vaccination | 1 | (41) | 4 |
| 7 | primary | 0 | | 4 |
| 8 | re-vaccination | 1 | (36) | 4 |

¹The number of previous vaccinations is followed by the number of years, separated by a comma if appropriate, between the previous vaccinations and the recent vaccination.
²The number of weeks between the most recent vaccination and the PBMC collection used to obtain CTL effectors.

### CTL Clones.

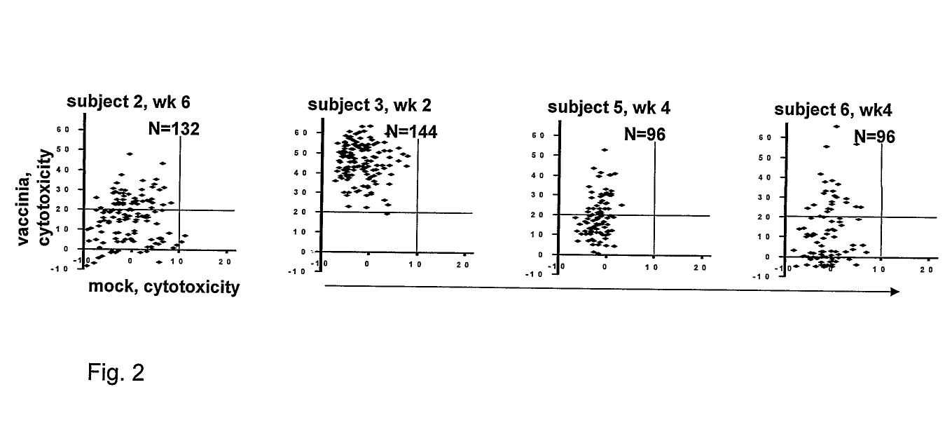

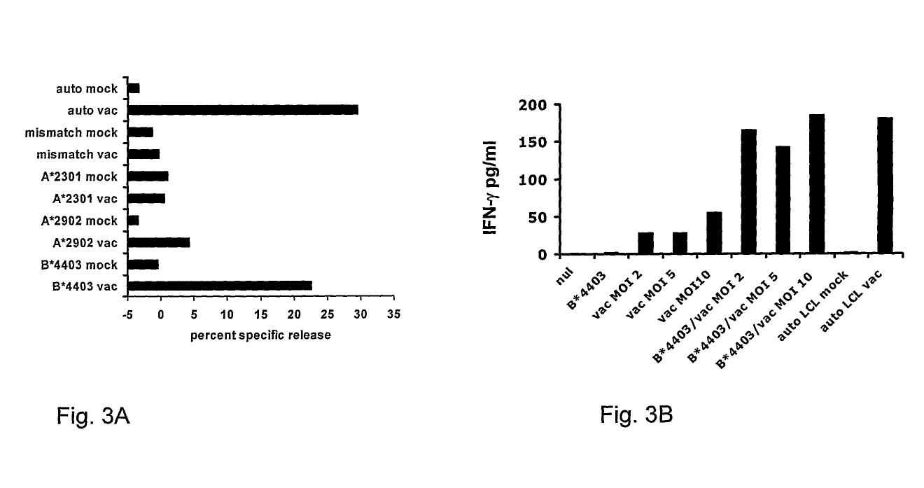

Panels of clones (96-144 per subject) were derived from bulk CD8 CTL from one primary and three revaccinees. From 27 to 99% of clones had vaccinia-specific CTL activity (FIG. 2) as defined in Materials and Methods. Clones with cytotoxicity and healthy microscopic appearance were expanded. From 85 to 100% of such clones proliferated briskly to anti-CD3 (26), were TCR αβ⁺, CD8+, and displayed vaccinia-specific lysis in confirmatory assays. After expansion, HLA class I A or B restriction was unambiguously assigned for most clones using both panels of partially matched APC and by vaccinia infection/HLA transfection assays (example, FIG. 3). Each clone investigated (n=5) gave identical results with both methods.

### Vaccinia Epitopes Recognized by HLAA*0101, B*0801, B*4403, A*2902, and A*2301 Restricted-CD8 CTL Clones

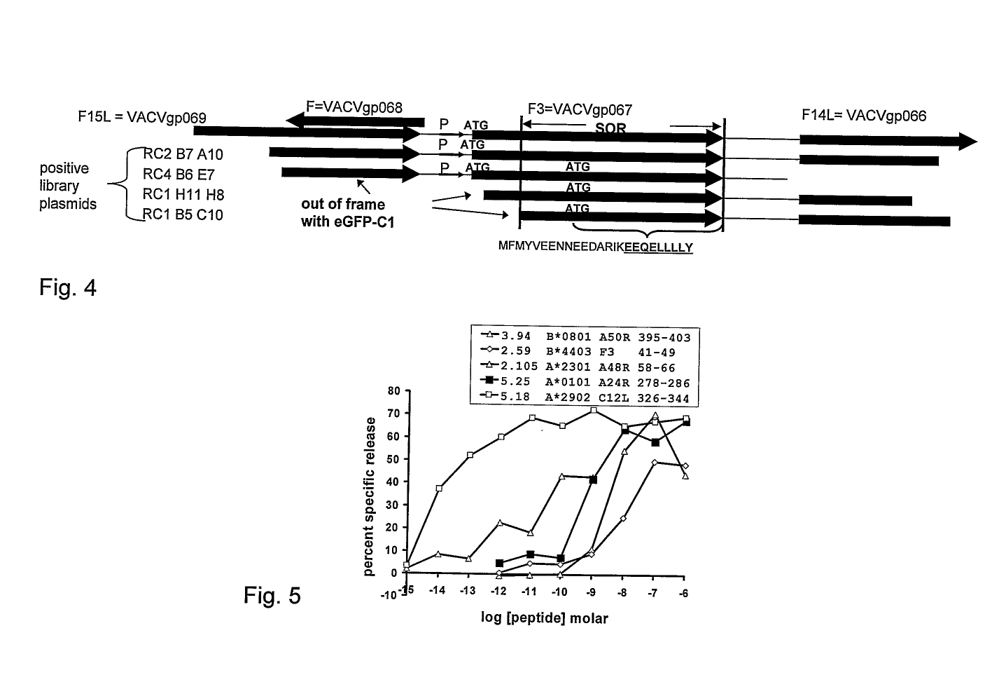

We defined peptide epitopes for five CD8 clones. For each, one or more vaccinia plasmids were strongly stimulatory for IFN-γ release, and only when cotransfected with the appropriate HLA cDNA. If multiple library hits were obtained, they were aligned and shortest overlapping regions (SOR) were determined. For example, the HLA B*4403-restricted clone 2.59 from a primary vaccinee yielded four independent library hits (FIG. 4). The SOR was the C-terminal 29 aa of the theoretical ORF F3. This 49-aa-long ORF (VACVgp067) is predicted to lie between ORFs F14L and F15L in vaccinia Copenhagen (GenBank NC_{—}001559), but has never been documented at the protein level. Of note, the plasmids RC4 B6 E7, RC1 H 11H8, and RC1 B5 C10 are fusions in which fragments of ORF F3, or the neighboring ORF F15 L, are predicted to be out of frame with eGFP. However, an ATG codon is present at predicted aa 25 of ORF F3. Sequence with features of a vaccinia early promoter (27), 5′ to the predicted initiation codon of F3, occurs in plasmids RC2 B7 A10 (and RC4 B6 E7). Full-length F3, cloned after PCR into pEGFP-C1 as an in-frame fusion, was positive in the IFN-γ Cos-7 cotransfection assay.

The candidate antigenic region, F3 25-49, was analyzed for peptides with the B*4403-binding motif (22). The peptide F3 41-49 (EEQELLLLY; SEQ ID NO: 34) was positive in CTL assays with an approximate EC50 of 10⁻⁸ molar (FIG. 5). It is likely that internal initiation or transcription from the vaccinia promoter occurred after transfection with the active genomic fragments. We previously documented internal ATG initiation and transcription/translation from viral promoters during similar library-based epitope discovery for HSV type 2 (HSV-2) (17). The presence of specific CD8 CTL in a vaccinia-infected human is the first documentation that F3 encodes a protein. F3 is highly conserved in orthopoxviruses (below), consistent with a role in replication or pathogenesis.

Similar overall strategies were used to discover four additional epitopes recognized by CD8 clones (FIG. 5). For each clone, HLA restriction was documented in CTL and transfection/infection assays. Each was similarly screened against the vaccinia genomic library, and positive pools were decoded to single active plasmids that were sequenced to identify antigenic fragments of the proteome. The translational schema for the active fragments of A50R recognized by a B*0801-restricted clone and fragments of A48R restricted by an A*2301-restricted clone were straightforward, entailing simple in-frame fusion of fragments of known vaccinia ORFS with eGFP. For an A*0101-restricted clone, the active fragments in ORF A24R were out-of-frame with a SOR covering aa 246-339; an internal ATG encoding methionine 256 proved to be upstream of the active epitope 278-286. Similarly, for an A*2902-restricted clone, the SOR of out-of-frame fragments of C12L was aa 301-353, with the eventual epitope 326-334 downstream of an internal ATG at methionine 320. Ag assignments were confirmed (Table II) with nonamet peptides in CTL assays (FIG. 5) with estimated EC50 values of 10⁻⁹ to 10⁻¹⁴ molar.

TABLE II

Peptides recognized by vaccinia-specific CD8 T-cells¹.

| | | | | | molluscum |
|---|---|---|---|---|---|
| | | | | | contagiousum |
| | | sequence | | | conservation⁴ |
| | | (SEQ ID NO: 22- | HLA | orthopox | (SEQ ID NO: 38-46, |
| ORF | epitope | 37, respectively) | allele | conservation² | respectively) |
| A3L | 90-98 | DEVASTHDW | B*4403 | primate OP all (+) | DEVASTQDW 8/9 |
| A3L | 264-272 | YEFRKYKSY | B*4403 | primate OP all (+) | YELKKVRPD 4/9 |
| A23R | 287-295 | HDVYGVSNF | B*4403 | primate OP all (+) | AHMYYGYHNF 5/9 |
| A24R | 278-286 | ITDFNIDTY | A*0101 | primate OP all (+) | EDDFDVAEY 3/9 |
| A48R | 58-66 | TYNDHIVNL | A*2301 | primate OP all (+) | no homolog |
| A50R | 395-403 | WLKIKRDYL | B*0801 | primate OP all (+) | no homolog |
| C12L | 326-334 | VYINHPFMY | A*2902 | fragmented in | no homolog |
| | | | | MVA (30) | |
| D1R | 126-134 | EERHIFLDY | B*4403 | primate OP all (+) | EEQYVFLDF 5/9 |
| D5R | 298-306 | LENGAIRIY | B*4403 | primate OP all (+) | LGNGALRIF 6/9 |
| D5R | 691-699 | EEIPDFAFY | B*4403 | primate OP all (+) | DLIPDFCFQ 5/9 |
| D5R | 349-357 | VWINNSWKF | A*2301 | primate OP all (+) | VWLRNCWRF 5/9 |
| E3L | 86-94 | DDVSREKSM | B*4403 | primate OP all (+) | no homolog |
| F3 | 41-49 | EEQELLLLY | B*4403 | primate OP all (+) | no homolog |
| I3L | 173-181 | IEGELESLS | B*4403 | primate OP all (+) | MLRELETLA 4/9 |
| IL- | 21-29 | DEIKCPNLN | B*4403 | Copen (−), MVA, | no homolog |
| 18bp³ | | | | variola, | |
| | | | | monkeypox (each | |
| | | | | divergent)) | |
| M2L | 38-46 | AELTIGYNY | B*4403 | deleted in NYVAC | no homolog |

¹CD8 CTL clones were tested in ⁵¹Cr release assays. Bulk CTL were tested for IFN-γ release and/or IFN-γ accumulation by ICC and only peptides with two or more positive tests are listed.
²Data from (22) and J. Tartaglia, personal communication. Primate orthopox (OP) analyzed were the primate orthopoxviruses vaccinia Copenhagen, vaccinia Western Reserve, MVA Acambis 3000, monkeypox (MP) Zaire, monkeypox Congo, variola major India, and NYVAC (25). (+) = peptide epitope predicted to be expressed, (−) = peptide epitope either altered or deleted and therefore not predicted to be expressed.
³The IL-18 binding protein is named, in vaccinia strain WR, VACWR013 and C12L (39). It is reported to be absent from Copenhagen (22). The epitope 21-29 is identical between vaccinia NYCBH and vaccinia WR, but is divergent in the homologous proteins in MVA, variola, and monkeypox.
⁴Data from (22). The sequence of the homologous region of the homologous protein, if any, is shown, followed by the number of identical amino acids at orthologous positions and the total number of amino acids.

### High-Throughput Expression Cloning

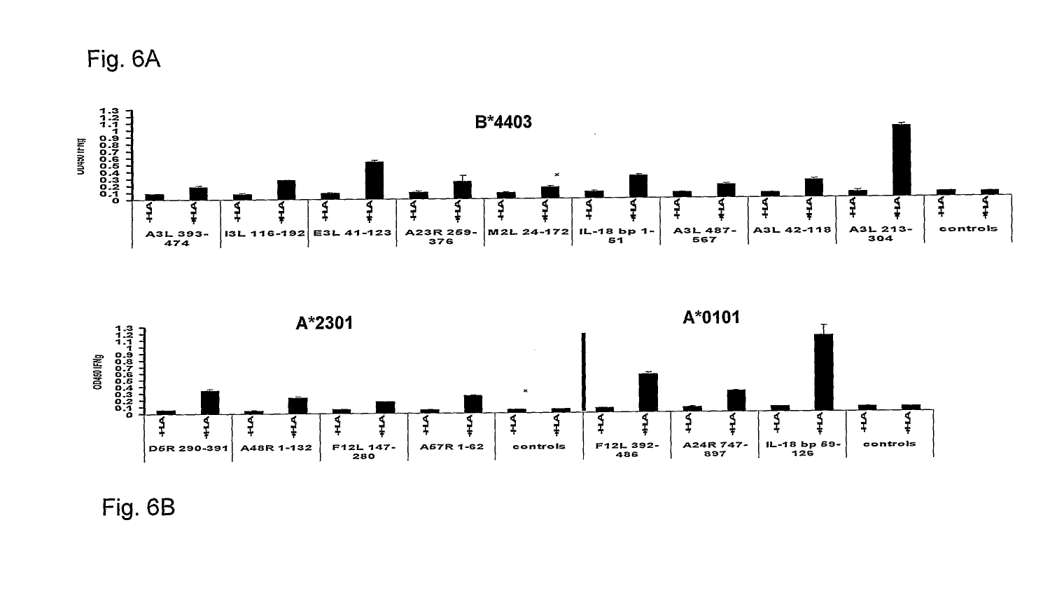

To speed epitope discovery, and explore within-subject diversity, we adapted expression cloning to bulk CTL responders. This eliminated the need to clone and expand CTL. A subset of the bulk CTL response was functionally isolated by transfecting Cos-7 cells with one of the subjects' HIA A or B alleles. We analyzed the B*4403- and A*2301-restricted repertoires of primary vaccinee subject 2, and the A*0101-restricted response of revaccinee subject 5. Bulk CTL typically yielded many positive pools of vaccinia genomic DNA with a gradation of IFN-γ responses. The pools stimulating the highest IFN-γ levels were broken down to identify single vaccinia genomic fragments that stimulate IFN-γ release when cotransfected with HLA cDNA (examples in FIG. 6). The biological activity of each positive vaccinia fragment reported was confirmed in at least one repeat assay.

Vaccinia sequences in active plasmids were assembled into contigs and compared with the vaccinia Copenhagen genome (21, 25). In addition, SOR (if applicable), internal ATG codons when appropriate, and the HLA-binding motif of the allele under study (22, 23) were used to select candidate peptide epitopes. These were synthesized and evaluated using IFN-γ ICC and/or ELISA to study peptide-level reactivity of bulk CTL. Each epitope in this report was positive in at least two repeats of one assay or one repeat of each assay.

Intracellular cytokine Cytometry

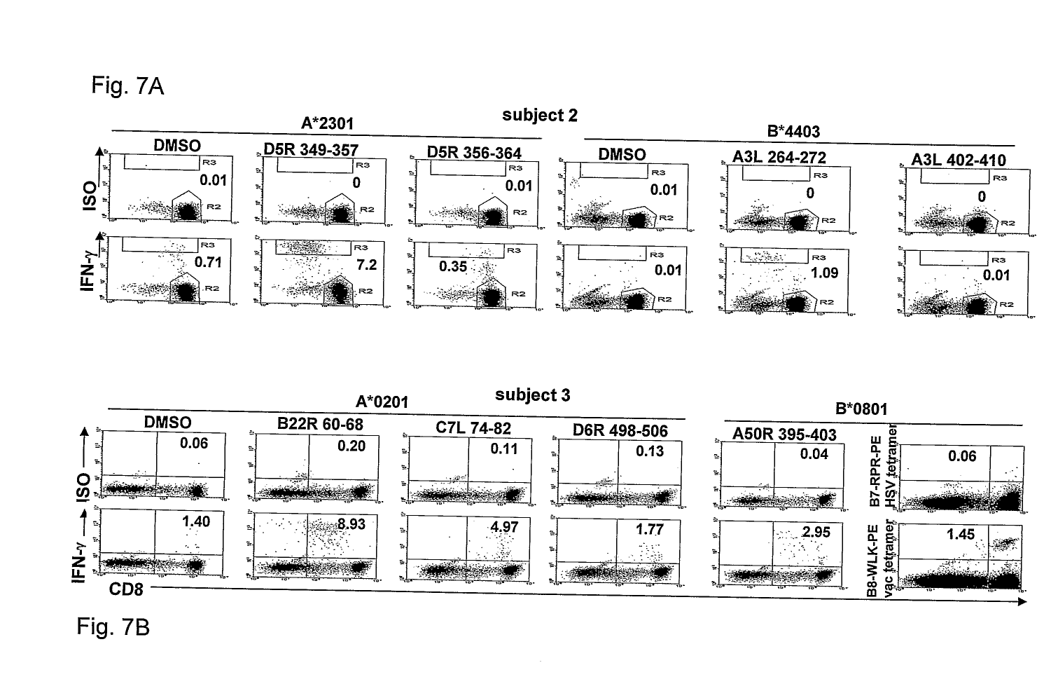

In the first format, single-cell IFN-γ responses were measured ICC after 15 h stimulation with vaccinia peptide (representative positive and negative examples and controls in FIG. 7). Responses in the presence of DMSO were somewhat above those observed with isotype control Ab, likely reflecting the prolonged (15 h) stimulation and residual activation from previous expansion and stimulation of the bulk CTL. Peptide stimulation lead to signals that were clearly separable from this background activation, ranging from 1.09 to 8.93%. The intensity of the specific IFN-γ signal was very bright, in contrast to the moderate intensity observed with DMSO control. Down-shift of CD8+ intensity was sometimes noted (see Discussion). ICC was used for rapid screening of candidate peptides. For example, a genomic fragment of ORF D5R encoding amino acids 290-391 was positive when cotransfected with A*2301 (FIG. 6). Peptide-binding algorithms high-affinity HLA B*2301 binding for both 349-357 and 356-364. These peptides were each tested and only 349-357 lead to detection of IFN-γ-bright cells at a level above the background seen with DMSO alone.

An additional application of ICC was measurement of the proportion of bulk CD8 CTL responsive to specific epitopes that were defined with CTL clones. For subject 3, 2.95% of bulk CTL recognized A50R 395-403, initially detected with clone 3.94. As 1.4% of cells responded to DMSO, the net response was ˜1.55%. Use of an HLA B*0801-A50R 395-403 tetramer to stain the same specimen detected 1.45% Ag-specific CD8 T cells (FIG. 7B), while a control HSV-2 tetramer (28) was negative.

### IFN-γ Secretion

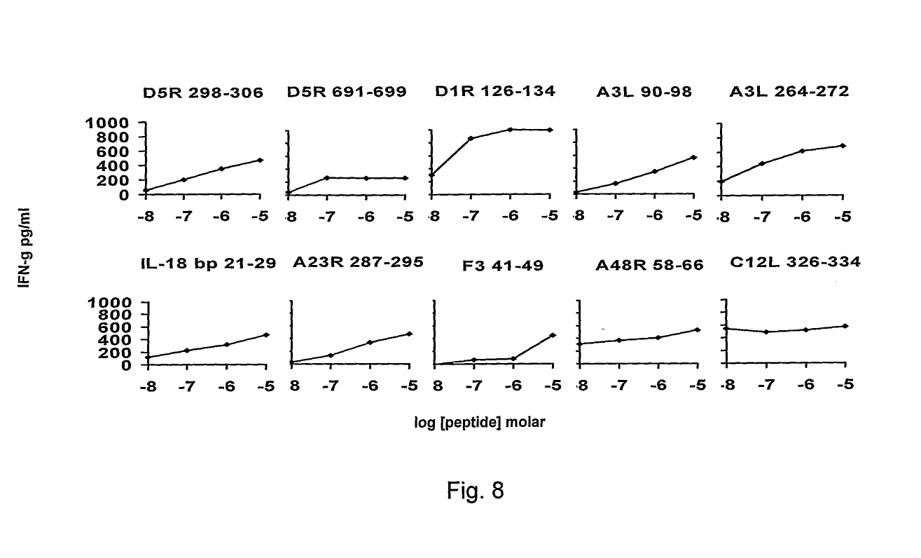

The second IFN-γ test format for high-throughput epitope discovery involved coincubation of bulk CTL with peptide-loaded autologous APC, and measurement of cytokine release into the media (FIG. 8). Most peptides checked were positive in both ICC and IFN-γ secretion tests (example, A3L 264-272, FIGS. 7 and 8), but IFN-γ secretion was generally more sensitive. For subject 2, eight additional epitopes (FIG. 8) were documented by IFN-γ release to lie within genornic fragments that were active upon cotransfection with HLA B*4403 (FIG. 6). Responses to the epitope in ORF F3 detected at the clonal level (FIGS. 4 and 5) were again detectable among bulk CTL. Of note, three discrete B*4403-restricted epitopes were detected in ORF A3L and two in ORF D5R. Overall, 16 epitopes have been defined by combining clonal reactivity and interrogation of bulk CTL with the IFN-γ ICC and secretion assays (Table II).

Seven additional vaccinia antigenic regions have been identified by cotransfection; definition of their internal peptide epitopes is still underway (Table III). These fragments have been repeatedly positive, contain regions of known ORFs, and are mostly straightforward, in-frame fusions with eGFP. Testing of candidate internal peptides is in progress. Of note, these data are consistent with the presence of additional epitopes in ORF A3L.

TABLE III
Regions of vaccinia ORFs that contain putative epitopes stimulating
human HLA class I-restricted CD8+ T-cells. Each fragment was
repeatedly positive after co-transfection with indicated HLA cDNA.
Recognition of an internal peptide has not yet been demonstrated.

| ORF¹ | HLA cDNA | predicted AA¹ |
|---|---|---|
| A3L | B*4403 | 487-567 |
| A3L | B*4403 | 393-474 |
| A24R | A*0101 | 747-897 |
| A57R | A*2301 | 1-62 |
| F12L | A*2301 | 147-280 |
| F12L | A*0101 | 392-486 |
| IL-18bp² | A*0101 | 59-126 |

¹Synthesis of data from sequence of biologically active plasmid(s). AA = amino acids. If more than one non-identical plasmid was recovered, the shortest overlapping region is reported. If one or more plasmids was out-of-frame with eGFP, internal AUG codons are considered the 5′ limit of regions possibly encoding an epitope or epitopes.
²The SOR was homologous to amino acids 59-126 of vaccinia WR VACWR013, also called C12L in this strain. The predicted vaccinia strain NYCBH 59-162 from our sequencing is divergent at the predicted amino acid level from homologous region of vaccinia WR.

The most detailed CD8 epitope data are available for subject 2, a primary vaccinee. The minimal estimate of the overall diversity of the CD8 response in this specimen is 18 epitopes. Specifically, for B*4403, 10 peptides stimulate bulk CTL (FIGS. 7 and 8), including one that stimulates a CD8 clone (FIGS. 5 and 8). Two additional nonredundant antigenic DNA regions, for which peptide identification is pending, also stimulate B*4403-restricted responses (Table III) for a total of at least 13 B*4403-restricted epitopes. Three antigenic DNA fragments contain epitopes restricted by A*2301 (Table III) that are nonredundant with clone 2.105 (FIG. 5) for a total of at least 4 A*2301-restricted epitopes. We also derived A*2902-restricted clones from this individual, increasing the diversity to at least 18.

HLA A*0201-restricted responses are of interest due to the high population prevalence of this allele. Subject 3, a revaccinee, had brisk HLA A*0201-restricted IFN-γ release by bulk CD8 CTL exposed to Cos-7 artificially transfected with A*0201 cDNA and infected with vaccinia. CD8+ clones with HLA A*0201-restricted CTL activity and IFN-γ release were also derived from this subject. For unknown reasons, discussed below, screening of the vaccinia genomic library for A*0201 epitopes was negative for both clonal and bulk CTL responders. We used the ICC assay to probe bulk CTL from this subject with five previously reported (11-13) A*0201-restricted epitopes (FIG. 7, bottom). Three peptides, B22R 60-68, C7L 74-82, and D6R 498-506, gave responses above background), while A26L 6-14 and H3L 184-192 did not.

### Discussion

The present example identifies vaccinia virus Ags and epitopes recognized by CD8 T cells in humans recently vaccinated with Dryvax. These results should be useful in comparing this replication-competent vaccine with other candidate products currently under evaluation for smallpox prevention. We have also made a prelirninary identification of candidate immunodominant Ags containing a high density of epitopes and gained an insight into the diversity of the response during acute infection. The contribution of responses to these epitopes to protection from orthopoxvirus challenge are unknown but could be addressed in challenge studies using HLA-transgenic animals after epitope-based vaccination.

This example describes 16 novel discrete epitopes within 15 vaccinia ORFs that are recognized in the context of four HLA class I alleles (HIA A*0101, A*2301, A*2902, and B*4403). HLA A*2301 belongs to the A24 supertype, while B*4403 belongs to the B44 supertype. A*0101 and related A*0101 supertype members are also prevalent in the population (29, 30). Although reactivity with other members of these supertypes will have to be studied empirically, the epitopes described in this report greatly extent published reports, limited to 5 epitopes restricted by A*0201 (11-13), and should allow monitoring of expanded patient cohorts. As almost all of the epitopes described in this report are conserved in MVA and NYVAC (Table IV), these epitopes should also be useful in monitoring the immune response to these replication-incompetent candidate vaccine strains.

TABLE IV
Selected virologic characteristics of novel human CD8 antigens
in vaccinia.

| ORF¹ | function² | kinetic class² |
|---|---|---|
| A3L | major core protein | late (40) |
| A23R | transcription factor | early (41) |
| A24R | DNA-dependent RNA polymerase | early-int-late (42) |
| A48R | thymidylate kinase/synthase | early (43) |
| A50R | DNA ligase, virulence | early (44) |
| A57R | guanylate kinase homologue (22) | unknown |
| C12L | serine protease inhibitor-like (45) | unknown |
| D1R | mRNA capping enzyme subunit (46) | early (47) |
| D5R | nucleoside triphosphatase, role in DNA | unknown |
| | replication (48) | |
| E3L | ss RNA binding, immune evasion (49) | early (50) |
| F3 | unknown; previously not documented to | unknown |
| | encode protein | |
| F12L | infectious enveloped virus protein; | early and late (51) |
| | extracellular enveloped virus formation, | |
| | virulence | |
| I3L | ss DNA binding protein (52) | early (53) |
| IL-18bp | immune evasion (54) | early (39) |
| M2L | unknown | early (55) |

¹Nomenclature from Hind III digest of vaccinia Copenhagen (26).
²Syntheses of data referenced and other reports, and texts (28).

A total of 16 distinct peptide epitopes recognized by human CD8 T cells were newly detected in this study. The conservation of epitopes between vaccine strains and pathogens is of interest for vaccine design. A summary of database (21) searches for epitope conservation in primate orthopoxviruses (Table II) indicates that most of the CD8 epitopes are identical in vatiola and monkeypox. Vaccinia MVA and NYVAC are replication-incompetent strains with deletions and disruptions of many ORFs (24). Almost all of the CD8 epitopes are predicted to be present in MVA and NYVAC, with the exception of Copenhagen M2L, which is not present in NYVAC (24), and C12L, which is fragmented in MVA (31). The epitope in the IL-18-binding protein, DEIKCPNLN (SEQ ID NO: 36), is identical in NYCBH and vaccinia WR. The homologous ORF is not present in vaccinia Copenhagen. Although predicted IL-18-binding proteins are present in MVA, variola, and monkeypox, the epitope region diverges at 2 or 3 aa (21). Specifically, the MVA and variola sequence is VETKCPNLD (SEQ ID NO:47), with changes at aa 1 and 3, and the monkeypox sequence is VETKCPNLA (SEQ ID NO: 48), with an additional change at the ninth residue. Most of the epitopes are present and identical in ectromelia, an orthopoxvirus of mice, but are divergent in canarypox, the backbone of the ALVAC vaccine vector (1), and molluscum contagiosum virus, a human pathogen (Table II). It has been speculated (27) that decreased smallpox vaccination may predispose individuals to molluscum contagiosum. The molluscum virus is only distantly related to vaccinia (27), and several of the antigenic vaccinia ORFs identified in this study do not have homologs in the molluscum virus (Table II). One epitope is relatively conserved (A3L 90-98 in vaccinia) at 8 of 9 aa, including anchor residues, but has a nonconservative difference at position 7. The other epitopes are quite divergent.

The virologic features of the vaccinia proteins newly identified as CD8 Ags in humans are diverse (Table IV). The known functions include enzymes, transcription factors, immune evasion proteins, and structural virion proteins. Of note, we have not detected epitopes in envelope proteins or in known targets of neutralizing Abs. Vaccinia genes are transcribed in several coordinated waves, designated early, intermediate, and late. Each kinetic class is immunogenic, with early proteins particularly well represented.

The determinants of immunodominance at the polypeptide level are largely unknown (32). We showed that several vaccinia ORFs contain multiple CD8 epitopes and are thus candidate immunodominant Ags. Specifically, A3L contains at least four epitopes (each B*4403 restricted), D5R at least three epitopes, and A24R, F12L, and IL-18-binding protein at least two epitopes each. These epitopes were discovered in independent iterations of an unbiased genome-wide screen, reducing the chance that epitope grouping is an artifact. Because the responder cells used in this report were studied after one cycle of expansion in response to live vaccinia virus, it is possible that some bias was introduced during restimulation that favored detection of some epitopes over others. The potential dominance of the vaccinia Ags mentioned above is testable by examination of subjects with diverse HLA type by ELISPOT or related techniques using short peptides from these ORFs.

The vaccinia Ags that were found to stimulate CD8 responses belonged to diverse functional and kinetic classes. Notably, viral regulatory and immune evasion genes and enzymes were wellrepresented, while we only detected one structural or envelope proteins that was a CD8 Ag (ORF A3L). None of the major neutralizing proteins (9) on infectious intracellular mature virion or extracellular-enveloped virion were targets of CDS T cells. Viral proteins synthesized at early times after infection were particularly well-represented. If cross-presentation is an important mode of Ag presentation for vaccinia-encoded Ags, as implied by some studies (33, 34), we would predict that abundant structural proteins would be better represented. We did not note any overlap at the ORF level with the ORFs previously reported to contain A*0201-restricted epitope, or with a set of ORFs recognized by CD8 T cells in mouse strain C57BL/6 (35). It is therefore likely that many additional antigenic ORFs remain to be uncovered, and that detailed analyses of many persons and HLA alleles will be required to assess the structural and kinetic correlates of CD8 antigenicity.

Our studies differ in several ways from other approaches to epitope discovery for complex viral pathogens. No knowledge of the viral genome sequence or predicted ORFS was used to generate our initial positive antigenic “hits”. The vaccinia genome was probed in an unbiased fashion and Ags were identified by library screening. Expression cloning should therefore be useful for studying T cell reactivity for unsequenced microbial pathogens or for identifying previously unsuspected ORFs. HLA peptide-binding motifs and algorithms were only used to define peptide epitopes within small (˜100 aa) antigenic fragments, and were not formally necessary, as the fragment size allows economical molecular truncation analyses and/or screening of internal peptides (19). Although peptide-binding motifs are known for some prevalent HLA alleles, HLA class I loci are extremely diverse, and reliance of these motifs for epitope discovery will exclude some HLA alleles from analysis.

The cells we probed for specificity by expression cloning are reactive with whole vaccinia, because they were studied after one cycle of in vitro expansion stimulated by live vaccinia. Our T cell clones, in addition, recognize vaccinia-infected cells in CTL assays. Both the clonal and bulk responders in our studies are documented to express CD8+. We used relatively low peptide concentrations in some assays (FIGS. 5 and 8). Taken together, these factors are consistent with the detection of vaccinia-specific CTL and decrease the likelihood of detection of cross-reactive T cells.

We initially validated our vaccinia library system using CTL clones (FIG. 5), as previously reported for HSV-2, but adapted the method to bulk-cultured CD8 CTL to speed epitope discovery. This variant offers higher throughput, but without loss of precision. Use of bulk CTL allowed rapid identification of antigenic genomic fragments (FIG. 6) and internal epitopes using IFN-γ release (FIG. 8) or ICC (FIG. 7). Overall, the “hit” rate for candidate peptides that we synthesized within antigenic genomic fragments was ˜70% for both cloned and bulk responder cells. This is far higher than the ˜1% rate obtained from bioinformatic scans of predicted ORFs and analyses of whole PBMC. In the ICC format, we noted bright, specific IFN-γ accumulation in CD8+int cells when some peptides were used. These cells are unlikely to be NK cells, as the responding bulk cultures are >98% TCR αβ⁺. Down-modulation of surface TCR αβ and associated molecules has been reported after activation through TCR (36). It is most likely that the IFN-γ^{high} cells in our ICC assays started as CD8^{high} cells and down-modulated surface CD8+ during our long (15 h) stimulation period.

As mentioned above, we were unable to score “hits” when screening HLA A*0201-restricted CTL clones, or bulk CTL lines with A*0201-restricted activity, using our genomic library. This was somewhat surprising, as bulk CTL reactivity was detected against known A*0201 epitopes (FIG. 7) in ORFS B22R, C7L, and D6R that do not have posttranslational modification, and should have been included in our library. The A*0201 expression plasmid was checked and protein expression was demonstrable in transfected Cos-7 cells. Assessment by PCR with primers spanning these epitopes should allow assessment of whether these epitopes are represented in our library and this type of analysis could be useful for quality control of next-generation libraries. Our analysis of the diversity of vaccinia-specific responses are not exhaustive, as a gradation of IFN-γ responses was detected when bulk CTL were detected against library pools, and not all positive pools have been decoded to single active plasmids (FIG. 6). We cannot yet determine whether we have detected the quantitatively most abundant responses within individuals, but the epitopes disclosed in this report should be useful for designing tetramer and peptide ELISPOT or ICC assays to examine this issue.

In summary, the human CD8 T cell response to vaccinia is robust at early times after vaccination. Expression cloning, including a new high-throughput variant, has disclosed that the response can be very diverse within an individual. Several candidate immunodominant Ags, containing multiple epitopes, have been described. These Ags and epitopes should be useful in developing candidate smallpox vaccines and modified poxvituses being developed as vectors for heterologous Ags.

### REFERENCES CITED IN EXAMPLE 1

1. Musey, L., Y. et al. 2003. J. Immunol. 171:1094-1101.
2. Redfield, R. R., et al. 1987. N. Engl. J. Med. 316:673-676.
3. Fulginiti, V. A., et al. 2003. Clin. Infect. Dis. 37:251-271.
4. Engler, R. J., et al. 2002. J. Allergy. Clin. Immunol. 110:357-365.
5. Karupiah, G., et al. 1996. J. Virol. 70:8301-8309.
6. Harrington, L. E., et al. 2002. J. Virol. 76:3329-3337.
7. Spriggs, M. K., et al. 1992. Proc. Natl Acad. Sci. U S A 89:6070-6074.
8. Xu, R., et al. 2004. J. Immunol. 172:6265-6271.
9. Edghill-Smith, Y., et al. 2005. Nat. Med. 11:740-747.
10. Hammarlund, E., et al. 2003. Nat. Med. 9:1131-1137.
11. Terajima, M., et al. 2003. J. Exp. Med. 197:927-932.
12. Drexler, I., et al. 2003. Proc. Natl. Acad. Sci. U S A 100:217-222.
13. Snyder, J. T., et al. 2004. J. Virol. 78:7052-7060.
14. Koelle, D. M. 2003. Methods 29:213-226.
15. Demkowicz, W. E., Jr., and F. A. Ennis. 1993. J. Virol. 67:1538-1544.
16. Koelle, D. M. 2003. Methods 29:213-226.
17. Tigges, M. A., et al. 1992. J. Virol 66:1622-1634.
18. Koelle, D. M., et al. 2001. J. Immunol. 166:4049-4058.
19. Posavad, C. M., et al. 2003. J. Immunol. 170:4380-4388.
20. Koelle, D. M., et al. 2003. Proc. Natl. Acad. Sci. U S A 100:12899-12904.
21. Koelle, D. M., et al. 2002. Blood 99:3844-3847.
22. Upton, C., et al. 2003. J. Virol 77:7590-7600.
23. Rammensee, H., et al. 1999. Immunogenetics 50:213-219.
24. Parker, K. C., et al. 1994. J. Immunol. 152:163-175.
25. Tartaglia, J., et al. 1992. Virology 188:217-232.
26. Goebel, S. J., et al. 1990. Virology 179:247-266, 517-263.
27. Brodie, S. J., et al. 1999. Nat. Med. 5:34-41.
28. Moss, B. 2001. In Fields Virology. P. M. Howley, editor. Lippincott Williams and Wilkins, Philadelphia. 2849-2883.
29. Koelle, D. M., et al. 2002. J. Clin. Invest. 110:537-548.
30. Antoine, G., et al. 1998. Virology 244:365-396.
31. Sette, A., and J. Sidney. 1998. Curr. Opinion Immunol. 10:478-482.
32. Marsh, S. G. E., et al. 2000. The HLA FactsBook. Academic Press, San Diego. 398 pp.
33. Yewdell, J. W., and M. Del Val. 2004. Immnunity 21:149-153.
34. Serna, A., et al. 2003. J. Immunol. 171:5668-5672.
35. Fonteneau, J. F., et al. 2003. Blood 102:4448-4455.
36. Tscharke, D. C., et al. 2005. J. Exp. Med. 201:95-104.
37. Niedergang, F., et al. 1997. J. Immunol. 159:1703-1710.
38. Staib, C., et al. 2005. J. Gen. Virol. 86:1997-2006.
39. Symons, J. A., et al. 2002. J. Gen. Virol. 83:2833-2844.
40. Rosel, J., and B. Moss. 1985. J. Virol. 56:830-838.
41. Sanz, P., and B. Moss. 1999. Proc. Natl Acad Sci. USA 96:2692-2697.
42. Patel, D. D., and D. J. Pickup. 1989. J. Virol. 63:1076-1086.
43. Hughes, S. J., 1991. J. Biol. Chem. 266:20103-20109.
44. Colinas, R. J., et al. 1990. Virology 179:267-275.
45. Senkevich, T. G., et al. 1993. Virus Res. 30:73-88.
46. Myette, J. R., and E. G. Niles. 1996. J. Biol. Chem. 271:11945-11952.
47. Morgan, J. R., et al. 1984. J. Virol. 52:206-214.
48. Beaud, G. 1995. Biochimie 77:774-779.
49. Langland, J. O., and B. L. Jacobs. 2004. Virology 324:419-429.
50. Yuwen, H., et al. 1993. Virology 195:732-744.
51. van Eijl, H., et al. 2002. J. Gen. Virol. 83:195-207.
52. Rochester, S. C., and P. Traktman. 1998. J. Virol. 72:2917-2926.
53. Welsch, S., et al. 2003. J. Virol. 77:6014-6028.
54. Reading, P. C., and G. L. Smith. 2003. J. Virol. 77:9960-9968.
55. Xiang, Y., et al. 1998. J. Virol. Z 72:7012-7023.

### EXAMPLE 2

### Additional Immunogenic Vaccinia Epitopes

This example demonstrates that there is a CD4+ T-cell response in humans to the vaccinia protein encoded by vaccinia genomic DNA that contains the vaccinia open reading frame (ORF) named L1R. The systematic name for this ORF is VACV COP 107 in the vaccinia strain Copenhagen genome (GenBank accession M35027) as published by Goebel S J et al, 1990, Virology 179: 247-266 and 517-563. There are several different ORF naming conventions for different strains of vaccinia. The term L1R refers to the ORF of this name in strain Copenhagen. The full length L1R protein has a predicted length of 250 amino acids. Our initial discovery process revealed that the fragment of L1R comprising amino acids 1-185 is immunologically active. This has been confirmed in multiple assays. We have followed this up by identifying an 11 amino acid long linear region of L1R that reacts with CD4+ T-cells. This epitope has the sequence KIQNVIIDECY (SEQ ID NO: 49), which represents amino acids 127-137.

We have also discovered that there is a CD4+ T-cell response in humans to the vaccinia protein encoded by vaccinia genomic DNA that contains the vaccinia open reading frame (ORF) named A33R. The systematic name for this ORF is VACV COP 191 in the vaccinia strain Copenhagen genome (GenBank accession M35027) as published by Goebel SJ et al, 1990, Virology 179: 247-266 and 517-563. The full length A33R protein has a predicted length of 185 amino acids. Our initial discovery process disclosed that the fragment of A33R comprising amino acids 58-185 is immunologically active. This has been confirmed in multiple assays. A 20 amino acid long linear region of A33R has been identified as containing the epitope, and has the sequence: NPITKTTSDYQDSDVSQEVR (SEQ ID NO: 50), corresponding to amino acids 157-176. This epitope region has been further narrowed down to the 14 amino acid-long sequence TKTTSDYQDSDVSQ (SEQ ID NO: 51), representing amino acids 160-173.

The sequence of the vaccinia ORF L1R and A33R proteins is very highly conserved between vaccinia and smallpox and monkeypox. Specifically, the full length monkeypox open reading frame L1R amino acid sequence and the smallpox sequence are 100% identical, as are the A33R amino acid sequences of monkeypox and smallpox. This makes it reasonable to assume that immunization with the vaccinia A33R or L1R protein would elicit a cross-reactive immune memory response that would also recognize smallpox and monkeypox virus. It is reasonable, as well, that many short or intermediate peptides within L1R or A33R will also elicit cross-reactive immunity. These fragments may be within or outside the particular fragments that we discovered contain a CD4 antigen.

### EXAMPLE 3

### Listing of Immunogenic Fragments

This example provides a listing of various fragments of vaccinia proteins that have been identified as immunogenic using the assays described above. The vaccinia strain NYCBH that was used for the methods described above is not sequenced. Most all of the sequences identified herein are 100% matches to Genbank sequences from strain Copenhagen, with the exception of IL-18bp, which is found in strain Western Reserve. The sequence of the IL-18bp-like protein used here is from NYCBH and the indicated amino acids 59-126 are:

(SEQ ID NO: 16)

RSDEDTKFIEHLGDGIKEDETVRTTDSGITTLRKVLHVTDTNKFAHYRF
TCVLTTIDGVSKKNIWLK.

| ORF | | | | | | | |
|---|---|---|---|---|---|---|---|
| Alpha- | | | | | | | |
| numeric | ORF | | Antigenically | | | | |
| name in | systemic | | active | | | Epitope | |
| vaccinia | name in | HLA | fragments | | Epitope | Sequence | |
| copen- | Vaccinia | restric- | discovered in | Shortest | Position for | (SEQ ID NO: | |
| hagen | Copenhagen | tion of | genetic | Overlapping | minimal 9- | 22-37, | Genbank |
| genome | genome | clone | screen* | Region* | mers | respectively) | No. |
| A3L | VACVgp154 | B4403 | AA 42-118 | AA 42-118 | AA 90-98 | DEVASTHDW | M35027 |
| A3L | VACVgp154 | B4403 | AA 273-304 | AA 273-304 | AA 264-272 | YEFRKVKSY | M35027 |
| A23R | VACVgp183 | B4403 | AA 259-376 | AA 259-376 | AA 287-295 | HDVYGVSNF | M35027 |
| A24R | VACVgp184 | A0101 | AA 108-338 | AA 246-338 | AA 278-286 | ITDFNIDTY | M35027 |
| | | | AA 246-480 | AA 256 “M” | | | |
| A48R | VACVgp217 | A2301 | AA 1-132 | AA 55-119 | AA 58-66 | TYNDHIVNL | M35027 |
| | | | AA 53-134 | | | | |
| | | | AA 55-119 | | | | |
| A50R | VACVgp219 | B0801 | AA 359-439 | AA 359-439 | AA 395-403 | WLKIKRDYL | M35027 |
| C12L | VACVgp018 | A2902 | AA 301-353 | AA 320-353 | AA 326-334 | VYINHPFMY | M35027 |
| | | | AA 320 “M” | | | | |
| D1R | VACVgp131 | B4403 | AA 47-158 | AA 47-158 | AA 126-134 | EERHIFLDY | M35027 |
| D5R | VACVgp138 | B4403 | AA 208-397 | AA 214-397 | AA 298-306 | LENGAIRIY | M35027 |
| | | | AA 214 “M” | | | | |
| D5R | VACVgp138 | B4403 | AA 606-760 | AA 618-760 | AA 691-699 | EEIPDFAFY | M35027 |
| | | | AA 618 “M” | | | | |
| D5R | VACVgp138 | A2301 | AA 290-391 | AA 290-391 | AA 349-357 | VWINNSWKF | M35027 |
| E3L | VACVgp075 | B4403 | AA 41-123 | AA 55-123 | AA 86-94 | DDVSREKSM | M35027 |
| | | | AA 55 “M” | | | | |
| F3 | VACVgp050 | B4403 | AA 25-49 | AA 25-49 | AA 41-49 | EEQELLLLY | M35027 |
| | | | AA 25 “M” | | | | |
| I3L | VACVgp093 | B4403 | AA 53-206 | AA 118-197 | AA 173-181 | IEGELESLS | M35027 |
| | | | AA 109-197 | | | | |
| | | | AA 118-257 | | | | |
| IL- | VACWR013 | B4403 | AA 1-41 | AA 1-41 | AA 21-29 | DEIKCPNLN | AY243312 |
| 18BP** | | | | | | | |
| M2L | VACVgp038 | B4403 | AA 24-172 | AA 24-172 | AA 38-46 | AELTIGVNY | M35027 |
| A3L | VACVgp154 | B4403 | AA 487-567 | | | | M35027 |
| A3L | VACVgp154 | B4403 | AA 393-474 | | | | M35027 |
| A24R | VACVgp184 | A0101 | AA 747-897 | | | | M35027 |
| A57R | VACVgp231 | A2301 | AA 1-62 | | | | M35027 |
| F12L | VACVgp063 | A2301 | AA 147-280 | | | | M35027 |
| F12L | VACVgp063 | A0101 | AA 392-486 | | | | M35027 |
| IL-18 | unknown | A0101 | AA 59-126 | | | | |
| bp like | | | | | | | |

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A pharmaceutical composition comprising an isolated vaccinia polypeptide, wherein the polypeptide comprises A3L, A23R, A24R, A33R, A48R, A50R, A57R, C12L, D1R, D5R, E3L, F3, F12L, I3L, IL-18bp, IL-18bp-like protein, L1R, or M2L, and a pharmaceutically acceptable carrier.

2. The pharmaceutical composition of claim 1, wherein the polypeptide comprises amino acids:
42-118 90-98, 213-304 273-304, 264-272, 392-474, 393-474, 487-567 of A3L;
259-376, 287-295 of A23R;
108-338, 267-339, 246-480, 246-339, 278-286, 747-897 of A24R;
160-173, 157-176, 58-185 of A33R;
58-66, 55-119, 55-120, 1-132, 1-133, 53-134, 54-136 of A48R;
395-403, 359-439 of A50R;
1-62 of A57R;
301-353, 326-334, 320-353 of C12L,
126-134, 47-158 of D1R;
208-397, 214-397, 349-357, 290-391, 298-306, 606-760, 618-760, 691-699 of D5R;
41-123, 55-123, 86-94 of E3L,
41-49, 149, 25-49, 26-49 of F3;
147-280, 392-386, 392-486 of F12L;
53-206, 109-197, 118-257, 118-197, 116-192, 173-181 of I3L;
1-41, 1-51, 21-29, 59-126 of IL-18bp;
59-126 of IL-18bp-like protein;
1-185, 127-137 of L1R,
24-172, or 38-46 of M2L.

3. The pharmaceutical composition of claim 1, wherein the polypeptide is a fusion protein.

4. The pharmaceutical composition of claim 3, wherein the fusion protein is soluble.

5. The pharmaceutical composition of claim 1, further comprising an adjuvant.

6. A polynucteotide that encodes a potypeptide set forth in claim 1.

7. A vector comprising the polynucleotide of claim 6.

8 A host cell transformed with the vector of claim 7.

9. A method of producing a vaccinia potypeptide comprising culturing the host cell of claim 8 and recovering the polypeptide so produced.

10. A vaccinia polypeptide produced by the method of claim 9.

11. A pharmaceutical composition comprising the polynucleotide of claim 6 and a pharmaceutically acceptable carrier

12. The pharmaceuticat composition of claim 11, further comprising an adjuvant.

13. A recombinant virus genetically modified to express an amino acid sequence consisting essentiaiy of amino acids as recited in claim 2.

14. The recombinant virus of claim 13 which is an adenovirus or alphavirus.

15. A pharmaceutical composition comprising the virus of claim 13 or and a pharmaceuticatly acceptable carrier.

16. The pharmaceutical composition of claim 15, further comprising an adjuvant.

17. A method of producing immune cells directed against vaccinia comprising contacting an immune cell with an antigen-presenting cell wherein the antigen-presenting cell is modified to present an epitope included in amino acids as recited in claim 2.

18. The method of claim 17, wherein the immune cell is a T cell.

19. The method of claim 18, wherein the T cell is a CD4+ or CD8+ T cell.

20. (canceled)

21. A method of killing a poxvirus infected celt comprising contacting an poxvirus-infected cell with the composition of claim 11.

22. (canceled)

23. (Canceled)

24. A method of enhancing production of poxvirus-specific antibody comprising contacting a poxvirus infected cell in a subject with the composition of claim 11.

25. A method of enhancing proliferation of poxvirus-specific T cells comprising contacting the poxvirus-specific T cells with an isolated polypeptide that comprises an epitope included in A3L, A23R, A24R, A33R, A48R, A50R, A57R, C12L, D1R, D5R, E3L, F3, F12L, I3L, IL-18bp, IL-18bp-like protein, L1R, or M2L of vaccinia virus.

26. A method of inducing an immune response to an poxvirus infection in a subject comprising administering the composition of claim 1 to the subject.

27. A method of treating a poxvirus infection in a subject comprising administering the composition of claim 1 to the subject.

28. A method of treating a poxvims infection in a subject comprising administering an antigen-presenting cell modified to present an epitope as recited in claim 2.
